(19) United States
(12) Patent Application Publication
(10) Pub. No.: US 2011/0020453 A1
(43) Pub. Date: Jan. 27, 2011

(54) Topical Formulations Comprising Ion Channel Modulators

(76) Inventors: Steven Blum, New York, NY (US); Barrett Katz, New York, NY (US); Christopher Wilfong, New York, NY (US); Brian Levy, New York, NY (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/702,064

(22) Filed: Feb. 8, 2010

Related U.S. Application Data
(60) Provisional application No. 61/150,665, filed on Feb. 6, 2009.

(51) Int. Cl. A61K 9/14; A61K 31/352; A61P 27/06

(52) U.S. Cl. 424/489; 514/456; 977/773; 977/788; 977/915

(57) ABSTRACT

Disclosed herein are pharmaceutical formulations comprising particles of formula 1:

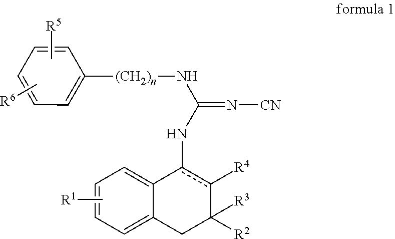

wherein R₁, R₂, R₃, R₄, R₅, R₆, n and * are each defined in specification. Also disclosed are methods of decreasing intraocular pressure comprising topically applying a benzopyranyl guanidine derivative of formula 1 to an eye.

### RELATED APPLICATIONS

This application claims priority to U.S. Provisional Application No. 61/150,665, filed on Feb. 6, 2009, which is hereby incorporated by reference in its entirety.

### BACKGROUND

Glaucoma, one of the leading causes of blindness, is an optic neuropathy associated with characteristic changes in the optic nerve. Elevated intraocular pressure is a significant risk factor for developing glaucoma and can damage the optic nerve. In humans, the optic nerve consists of millions of axons from neurons whose perikarya reside primarily in the ganglion cell layer and, to a lesser extent, in the inner part of the inner nuclear layer. The excavated appearance of the optic nerve head in glaucoma is thought to be caused by the death and subsequent loss of ganglion cells and their axons [N. N. Osborne, et. Al. Survey of Ophthalmology, 43; suppl. S102-s128 (1999)].

Benzopyranyl guanidine derivatives represented by formula 1 below have been shown to provide superior cardioprotective and neuroprotective activity from ischemia-reperfusion and hypoxic damage, for example, as described in U.S. Pat. No. 6,323,238, which is incorporated herein by reference in its entirety. The compounds also are thought to exhibit various pharmacological efficacies, including prevention of lipid peroxidation and reactive oxygen species formation, protection of ischemic retina, improvement of impaired nociceptive responses in diabetic rats, inhibition of NO formation, and suppression of angiogenesis and restenosis.

The present inventors have discovered methods of applying formulations, including a benzopyranyl guanidine derivative represented by the formula 1, topically to an eye to reduce intraocular pressure in the eye.

### DETAILED DESCRIPTION

One embodiment of the invention relates to a topical pharmaceutical formulation, comprising:

a compound of formula 1:

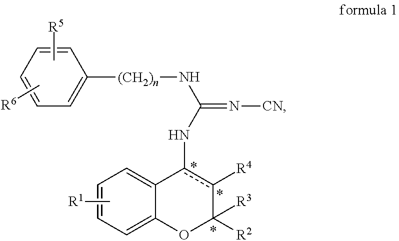

or a pharmaceutically acceptable salt thereof, wherein:

R¹ represents H, halogen, CF₃, NO₂, CN, OR^{a}, O(C═O)R^{a}, COOR^{a}, NH₂, NHS(O)ₘR^{a}, NH(C═O)R^{a} or S(O)ₘR^{a}; R^{a} represents straight or branched alkyl group of C₁-C₄ or aryl group; and m is an integer of 0-2,

R² represents straight or branched alkyl group of C₁-C₄,

R³ represents CH₂OR^{a},

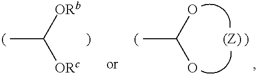

R^{a} is defined as above; R^{b} and R^{c} are independent of each other and represent a straight or branched alkyl group of C₁-C₄, respectively; and Z represents a straight or branched alkyl group of C₁-C₅,

R⁴ represents OH, H, halogen, ONO₂ or O(C═O)R^{a}; and R^{a} is defined as above;

R⁵ and R⁶ are independent of each other and represent H, halogen, straight or branched alkyl group of C₁-C₃, OR^{a}, CX₃, NO₂, CO₂Rₐ, —(C═O)Rₐ or SO₃R^{a}; R^{a} is defined as above; and X represents halogen,

n is an integer of 0-2,

and * represents the chiral center;

wherein the compound of formula 1 is provided as microparticles or nanoparticles having an average diameter of about 0.1 micron to about 20 microns; and

a pharmaceutically acceptable carrier comprising a solubilizing agent; a surfactant, a buffer, a chelator, a suspending agent, an isotonizer, a preservative, a stabilizing agent, a thickening agent, a pH adjusting agent, or mixtures thereof.

The formulation may include the compound of formula 1 in an amount of more than 0% to an amount of 100% by weight. In one embodiment, the compound of formula 1 may be present in the formulation in an amount of about 0.5% to about 30%, about 1% to 30%, about 1% to 20%, about 1% to about 15%, or about 1 to about 10%. In other embodiments, the compound of formula 1 is an amount of about 3% to about 10% of the formulation. In another aspect, the compound of formula 1 may be present in about 1, %, about 2%, about 3%, about 4%, about 5%, about 6%, about 7%, about 8%, about 9%, or about 10% by weight.

In some embodiments, the formulation comprises the freebase of a compound of formula 1.

Other embodiments may include formulations comprising the solvates and hydrates which can be prepared from benzopyranyl guanidine derivatives of formula 1 in addition to benzopyranyl guanidine derivatives of formula 1 and their pharmaceutically acceptable salts. For example, the compounds of formula 1 may be provided as acceptable salts derived from pharmaceutically or physiologically acceptable free acids. These salts include but are not limited to the following: angiogenesis salts with inorganic acids such as hydrochloric acid, hydrobromic acid, sulfuric acid, sulfonic acid, phosphoric acid, stannic acid, etc. and organic acids such as citric acid, acetic acid, lactic acid, maleic acid, fumaric acid, gluconic acid, methanesulfonic acid, glycolic acid, succinic acid, tartaric acid, 4-toluenesulfonic acid, galacturonic acid, embonic acid, glutamic acid, and aspartic acid, etc.

The acid salts of the compounds of formula 1 can be prepared in any suitable manner, for example by dissolving the compounds of the various aspects in excess aqueous acid and precipitating the salt with a water-miscible organic solvent, such as methanol, ethanol, acetone or acetonitrile. The acidic salts may be prepared by heating equivalent amounts of the compounds and an acid in water or an alcohol, such as glycol monomethyl ether, and then evaporating the mixture to dryness or filtering off the precipitated salt with suction.

In addition, the compounds of formula 1 may be in the form of pharmaceutically acceptable ammonium, alkali metals or alkaline earth metals salts. The alkali metal or alkaline earth metal salts can be obtained, for example, by dissolving the compound of formula 1 in equimolar amount of alkali metal or alkaline earth metal hydroxide solution, filtering from the undissolved materials and evaporating the filtrate to dryness. Sodium, potassium or calcium salts, among others, are pharmaceutically suitable. The corresponding silver salts may be obtained by the reaction of an alkali metal or alkaline earth metal salt with a suitable silver salt, such as silver nitrate.

Further embodiments include the separate stereochemical isomers, i.e. diastereomerically pure or enantiomerically pure compounds which have one or more chiral centers at 2, 3 and 4-positions, in addition to the racemic mixtures or diastereomeric mixtures of benzopyranyl guanidine derivatives of formula 1.

In embodiments having three chiral centers at 2, 3 and 4-positions, the 3,4-dihydro benzopyran derivatives are represented by the optical isomers such as (I₁), (I₂), (I₃) and (I₄) (See the following formula 3),

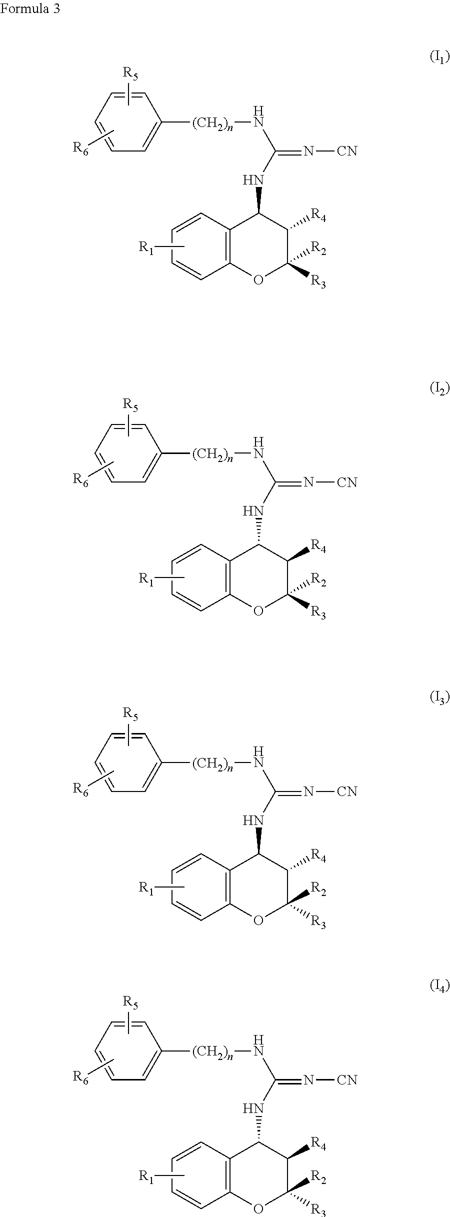

wherein R¹, R², R³, R⁴, R⁵, R⁶ and n are defined as above.

Another embodiment of the invention relates to formulations comprising a compound of formula 1 as defined above, wherein: R¹ represents NO₂, CN, NH₂ or S(O)ₘR^{a}; R^{a} represents straight or branched alkyl group of C₁-C₂, or an aryl group; and m is an integer of 0-2,

R² represents CH₃,

R³ represents

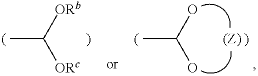

R^{b} and R^{c} are independent of each other and represent straight or branched alkyl groups of C₁-C₃, respectively; and Z represents straight or branched alkyl group of C₁-C₅,

R⁴ represents OH, H or O(C═O)R^{a}; and R^{a} represents a straight or branched alkyl group of C₁-C₃;

R⁵ and R⁶ are independent of each other and represent H, halogen, straight or branched alkyl groups of C₁-C₃, OR^{a}, CX₃ or NO₂; R^{a} represents a straight or branched alkyl group of C₁-C₃; and X represents halogen, and

n is an integer of 0-2.

In another embodiment, the compound of formula 1 is selected from the group consisting of:

1) (2R,3R,4S)-N″-cyano-N-(6-nitro-3,4-dihydro-3-hydroxy-2-methyl-2-dimethoxymethyl-2H-benzopyran-4yl)-N′-(4-chlorophenyl)guanidine;

2) (2R,3S,4R)-N″-cyano-N-(6-nitro-3,4-dihydro-3-hydroxy-2-methyl-2-dimethoxymethyl-2H-benzopyran-4-yl)-N′-(4-chlorophenyl)guanidine;

3) (2R,3R,4S)-N″-cyano-N-(6-nitro-3,4-dihydro-3-hydroxy-2-methyl-2-dimethoxymethyl-2H-benzopyran-4-yl)-N′-(3-chlorophenyl)guanidine;

4) (2R,3S,4R)-N″-cyano-N-(6-nitro-3,4-dihydro-3-hydroxy-2-methyl-2-dimethoxymethyl-2H-benzopyran-4-yl)-N′-(3-chlorophenyl)guanidine;

5) (2R,3R,4S)-N″-cyano-N-(6-nitro-3,4-dihydro-3-hydroxy-2-methyl-2-dimethoxymethyl-2H-benzopyran-4-yl)-N′-(4-nitrophenyl)guanidine;

6) (2R,3R,4S)-N″-cyano-N-(6-nitro-3,4-dihydro-3-hydroxy-2-methyl-2-dimethoxymethyl-2H-benzopyran-4-yl)-N′-(3-trifluoromethylphenyl)guanidine;

7) (2R,3S,4R)-N″-cyano-N-(6-nitro-3,4-dihydro-3-hydroxy-2-methyl-2-dimethoxymethyl-2H-benzopyran-4-yl)-N′-(3-trifluoromethylphenyl)guanidine;

8) (2R,3R,4S)-N″-cyano-N-(6-nitro-3,4-dihydro-3-hydroxy-2-methyl-2-dimethoxymethyl-2H-benzopyran-4-yl)-N′-(4-methoxyphenyl)guanidine;

9) (2R,3S,4R)-N″-cyano-N″-6-nitro-3,4-dihydro-3-hydroxy-2-methyl-2-dimethoxymethyl-2H-benzopyran-4yl)-N′-(4-methoxyphenyl)guanidine;

10) (2S,3R,4S)-N″-cyano-N-(6-nitro-3,4-dihydro-3-hydroxy-2-methyl-2-dimethoxymethyl-2H-benzopyran-4-yl)-N′-(4-chlorophenyl)guanidine;

11) (2S,3S,4R)-N″-cyano-N-(6-nitro-3,4-dihydro-3-hydroxy-2-methyl-2-dimethoxymethyl-2H-benzopyran-4-yl)-N′-(4-chlorophenyl)guanidine;

12) (2S,3R,4S)-N″-cyano-N-(6-nitro-3,4-dihydro-3-hydroxy-2-methyl-2-dimethoxymethyl-2H-benzopyran-4-yl)-N′-(3-chlorophenyl)guanidine;

13) (2S,3S,4R)-N″-cyano-N-(6-nitro-3,4-dihydro-3-hydroxy-2-methyl-2-dimethoxymethyl-2H-benzopyran-4-yl)-N′-(3-chlorophenyl)guanidine;

14) (2S,3R,4S)-N″-cyano-N-(6-nitro-3,4-dihydro-3-hydroxy-2-methyl-2-dimethoxymethyl-2H-benzopyran-4-yl)-N′-(3-trifluoromethylphenyl)guanidine;

15) (2S,3S,4R)-N″-cyano-N-(6-nitro-3,4-dihydro-3-hydroxy-2-methyl-2-dimethoxymethyl-2H-benzopyran-4-yl)-N′-(3-trifluoromethylphenyl)guanidine;

16) (2S,3R,4S)-N″-cyano-N-(6-nitro-3,4-dihydro-3-hydroxy-2-methyl-2-dimethoxymethyl-2H-benzopyran-4-yl)-N′-(4-methoxyphenyl)guanidine;

17) (2S,3S,4R-N″-cyano-N-(6-nitro-3,4dihydro-N′-(4-methoxyphenyl)guanidine;

18) (2R,3R,4S)-N″-cyano-N-(6-nitro-3,4-dihydro-3-hydroxy-2-methyl-2-dimethoxymethyl-2H-benzopyran-4-yl)-N′-(4-methylphenyl)guanidine;

19) (2R,3S,4R)-N″-cyano-N-(6-nitro-3,4-dihydro-3-hydroxy-2-methyl-2-dimethoxymethyl-2H-benzopyran-4-yl)-N′-(4-methylphenyl)guanidine;

20) (2R,3R,4S)-N″-cyano-N-(6-nitro-3,4-dihydro-3-hydroxy-2-methyl-2-dimethoxymethyl-2H-benzopyran-4-yl)-N′-(4-methoxybenzyl)guanidine;)

21) (2R,3S,4R)-N″-cyano-N-(6-nitro-3,4-dihydro-3-hydroxy-2-methyl-2-dimethoxymethyl-2H-benzopyran-4-yl)-N′-(4-methoxybenzyl)guanidine;

22) (2R,3R,4S)-N″-cyano-N-(6-nitro-3,4-dihydro-3-hydroxy-2-methyl-2-dimethoxymethyl-2H-benzopyran-4-yl)-N′-benzylguanidine;

23) (2R,3S,4R)-N″-cyano-N-(6-nitro-3,4-dihydro-3-hydroxy-2-methyl-2-dimethoxymethyl-2H-benzopyran-4-yl)-N′-benzylguanidine;

24) (2S,3S,4R)-N″-cyano-N-(6-nitro-3,4-dihydro-3-hydroxy-2-methyl-2-dimethoxymethyl-2H-benzopyran-4-yl)-N′-benzylguanidine;

25) (2S,3S,4R)-N″-cyano-N-(6-nitro-3,4-dihydro-3-hydroxy-2-methyl-2-dimethoxymethyl-2H-benzopyran-4yl)-benzylguanidine;

26) (2R,3R,4S)-N″-cyano-N-(3,4-dihydro-3-hydroxy-2-methyl-2-dimethoxymethyl-2H-benzopyran-4-yl)-N′-(4-chlorophenyl)guanidine;

27) (2R,3S,4R)-N″-cyano-N-(3,4-dihydro-3-hydroxy-2-methyl-2-dimethoxymethyl-2H-benzopyran-4-yl)-N′-(4-chlorophenyl)guanidine;

28) (2R,3R,4S)-N″-cyano-N-(6-nitro-3,4-dihydro-3-hydroxy-2-methyl-2-hydroxymethyl-2H-benzopyran-4-yl)-N′-(4-chlorophenyl)guanidine;

29) (2R,3S,4R)-N″-cyano-N-(6-nitro-3,4-dihydro-3-hydroxy-2-methyl-2-hydroxymethyl-2H-benzopyran-4-yl)-N′-(4-chlorophenyl)guanidine;

30) (2R,3R,4S)-N″-cyano-N-(6-nitro-3,4-dihydro-3-hydroxy-2-methyl-2-methoxymethyl-2H-benzopyran-4-yl)-N′-(4-chlorophenyl)guanidine;

31) (2R,3S,4R)-N″-cyano-N-(6-nitro-3,4-dihydro-3-hydroxy-2-methyl-2-methoxymethyl-2H-benzopyran-4-yl)-N′-(4-chlorophenyl)guanidine;

32) (2S,3R,4S)-N″-cyano-N-(6-nitro-3,4-dihydro-3-hydroxy-2-methyl-2-dimethoxymethyl-2H-benzopyran-4-yl)-N-(2-chlorophenyl)guanidine;

33) (2S,3S,4R)-N″-cyano-N-(6-nitro-3,4dihydro-3-hydroxy-2-dimethoxymethyl-2H-benzopyran-4-yl)-N′-(2-chlorophenyl)guanidine;

34) (2S,3R,4S)-N″-cyano-N-(6-nitro-3,4-dihydro-3-hydroxy-2-methyl-2-dimethoxymethyl-2H-benzopyran-4-yl)-N′-(2-trifluoromethylphenyl)guanidine;

35) (2S,3S,4R)-N″-cyano-N-(6-nitro-3,4-dihydro-3-hydroxy-2-methyl-2-dimethoxymethyl-2H-benzopyran-4-yl)-N′-(2-trifluoromethylphenyl)guanidine;

36) (2S,3S,4S)-N″-cyano-N-(6-nitro-3,4-dihydro-3-hydroxy-2-methyl-2-dimethoxymethyl-2H-benzopyran-4-yl)-N′-(2-chlorobenzyl)guanidine;

37) (2S,3S,4R)-N″-cyano-N-(6-nitro-3,4-dihydro-3-hydroxy-2-methyl-2-dimethoxymethyl-2H-benzopyran-4-yl)-N′-(2-chlorobenzyl)guanidine;

38) (2S,3S,4R)-N″-cyano-N-(6-nitro-3,4-dihydro-3-acetoxy-2-methyl-2-dimethoxymethyl-2H-benzopyran-4-yl)-N′-benzylguanidine;

39) (2S)-N″-cyano-N-(6-nitro-2-methyl-2-dimethoxymethyl-2-H-benzopyran-4-yl)-N′-benzylguanidine;

40) (2S,3S,4R)-N″-cyano-N-(6-amino-3,4-dihydro-3-hydroxy-2-methyl-2-dimethoxymethyl-2H-benzopyran-4-yl)-N″-benzylguanidine;

41) (2S,3S,4R)-N″-cyano)-N-(6-acetoxyamino-3,4-dihydro-3-hydroxy-2-methyl-2-dimethoxymethyl-2H-benzopyran-4-yl)-N′-benzylguanidine;

42) (2S,3S,4R)-N″-cyano-N-(6-methanesulfonylamino-3,4-dihydro-3-hydroxy-2-methyl-2-dimethoxymethyl-2H-benzopyran-4-yl)-N′-benzylguanidine;

43) (2S,3S,4R)-N″-cyano-N-(6-cyano-3,4-dihydro-3-hydroxy-2-methyl-2-dimethoxymethyl-2H-benzopyran-4-yl)-N′-(4-chlorophenyl)guanidine;

44) (2S,3R,4S)-N″-cyano-N-(6-cyano-3,4-dihydro-3-hydroxy-2-methyl-2-dimethoxymethyl-2H-benzopyran-4-yl)-N′-(4-chlorophenyl)guanidine;

45) (2S,3S,4R)-N″-cyano-N-(6-cyano-3,4-dihydro-3-hydroxy-2-methyl-2-dimethoxymethyl-2H-benzopyran-4-yl)-N′-benzylguanidine;

46) (2S,3R,4S)-N″-cyano-N-(6-cyano-3,4-dihydro-3-hydroxy-2-methyl-2-dimethoxymethyl-2H-benzopyran-4-yl)-N′-benzylguanidine;

47) (2S,3S,4R)-N″-cyano-N-(6-bromo-3,4-dihydro-3-hydroxy-2-methyl-2-dimethoxymethyl-2H-benzopyran-4-yl)-N′-benzylguanidine;

48) (2S,3S,4R)-N″-cyano-N-(6-nitro-3,4-dihydro-3-hydroxy-2-methyl-2-(dimethoxymethyl-2H-benzopyran-4-yl)-N′-(3,4-dimethoxybenzyl)guanidine;

49) (2S,3S,4R)-N″-cyano-N-(6-amino-3,4-dihydro-3-hydroxy-2-methyl-2-(dimethoxymethyl-21-benzopyran-4-yl)-N′-(3,4-dimethoxylbenzyl)guanidine;

50) (2S,3S,4R)-N″-cyano-N-(6-nitro-3,4-dihydro-3-hydroxy-2-methyl-2-dimethoxymethyl-2H-benzopyran-4-yl)-N′-(4-methoxybenzyl)guanidine;

51) (2S,3S,4R)-N″-cyano-N-(6-amino-3,4-dihydro-3-hydroxy-2-methyl-2-dimethoxymethyl-2H-benzopyran-4-yl)-N′-(4-methoxybenzyl)guanidine;

52) (2S,3S,4R)-N″-cyano-N-(6-nitro-3,4-dihydro-3-hydroxy-2-methyl-2-dimethoxymethyl-2H-benzopyran-4-yl)-N′-(3-nitrobenzyl)guanidine;

53) (2S,3S,4R)-N″-cyano-N-(6-nitro-3,4-dihydro-3-hydroxy-2-methyl-2-dimethoxymethyl-2H-benzopyran-4-yl)-N′-(3-trifluoromethylbenzyl)guanidine;

54) 2S,3S,4R)-N″-cyano-N-(6-amino-3,4-dihydro-3-hydroxy-2-methyl-2-dimethoxymethyl-2H-benzopyran-4-yl)-N′-(3-trifluoromethylbenzyl)guanidine;

55) (2S,3S,4R)-N″-cyano-N-(6-methanesulfonyloxy-3,4-dihydro-3-hydroxy-2-methyl-2-dimethoxymethyl-2H-benzopyran-4-yl)-N′-benzylguanidine;

56) (2R,3S,4R)-N″-cyano-N-(6-methanesulfonyloxy-3,4-dihydro-3-hydroxy-2-methyl-2-dimethoxymethyl-2H-benzopyran-4-yl)-N′-benzylguanidine;

57) (2S,3R,4S)-N″-cyano-N-(6-amino-3,4-dihydro-3-hydroxy-2-methyl-2-dimethoxymethyl-2!!-benzopyran-4-yl)-N′-benzylguanidine;

58) (2R,3R,4S)-N″-cyano-N-(6-amino-3,4-dihydro-3-hydroxy-2-methyl-2-dimethoxymethyl-2H-benzopyran-4-yl)-N′-benzylguanidine;

59) (2R,3S,4R)-N″-cyano-N-(6-amino-3,4-dihydro-3-hydroxy-2-methyl-2-dimethoxymethyl-2H-benzopyran-4-yl)-N′-benzylguanidine;

60) (2S,3S,4R)-N″-cyano-N-(6-nitro-3,4-dihydro-3-hydroxy-2-methyl-2-([1,3]dioxolan-2-yl)-2H-benzopyran-4-yl)-N′-benzylguanidine;

61) (2S,3S,4R)-N″-cyano-N-(6-amino-3,4-dihydro-3-hydroxy-2-methyl-2-([1,3]dioxolan-2-yl)-2H-benzopyran-4-yl)-N-benzylguanidine;

62) (2S,3S,4R)-N″-cyano-N-(6-nitro-3,4-dihydro-3-hydroxy-2-methyl-2-([1,3]dioxan-2-yl)-2H-benzopyran-4-yl)-N′-benzylguanidine;

63) (2S,3S,4R)-N″-cyano-N-(6-amino-3,4-dihydro-3-hydroxy-2-methyl-2-([1,3]dioxan-2-yl)-2H-benzopyran-4-yl)-N′-benzylguanidine;

64) (2S,3S,4R)-N″-cyano-N-(6-nitro-3,4-dihydro-3-hydroxy-2-methyl-2-([1,3]-5,5-dimethyldioxan-2-yl)-2H-benzopyran-4-yl)-N′-benzylguanidine;

65) (2S,3S,4R)-N″-cyano-N-(6-amino)-3,4-dihydro-3-hydroxy-2-methyl-2-([1,3]-5,5-dimethyldioxan-2-yl)-2H-benzopyran-4-y)-N′-benzylguanidine;

66) (2S,3S,4R)-N″-cyano-N-(6-nitro-3,4-dihydro-3-hydroxy-2-methyl-2-diethoxymethyl-2H-benzopyran-4-yl)-N′-benzylguanidine;

67) (2S,3S,4R)-N″-cyano-N-(6-amino-3,4-dihydro-3-hydroxy-2-methyl-2-diethoxymethyl-2H-benzopyran-4-yl)-N′-benzylguanidine;

68) (2S,3S,4R)-N″-cyano-N-(6-methoxycarbonyl-3,4-dihydro-3-hydroxy-2-methyl-2-dimethoxymethyl-2H-benzopyran-4-yl)-N′-benzylguanidine;

69) (2R,3S,4R)-N″-cyano-N-(6-methoxycarbonyl-3,4-dihydro-3-hydroxy-2-methyl-2-dimethoxymethyl-2H-benzopyran-4-yl)-N′-benzylguanidine;

70) (3S,4R)-N″-cyano-N-(8-nitro-3,4-dihydro-3-hydroxy-2-methyl-2-dimethoxymethyl-2H-benzopyran-4-yl)-N′-benzylguanidine;

71) (2S,3S,4R)-N″-cyano-N-(8-amino-3,4-dihydro-3-hydroxy-2-methyl-2-dimethoxymethyl-2H-benzopyran-4-yl)-N′-benzylguanidine;

72) (2R,3S,4R)-N″-cyano-N-(8-amino-3,4-dihydro-3-hydroxy-2-methyl-2-dimethoxymethyl-2H-benzopyran-4-yl)-N′-benzylguanidine;

73) (2S,3S,4R)-N″-cyano-N-(6-amino-3,4-dihydro-3-hydroxy-2-methyl-2-dimethoxymethyl-2H-benzopyran-4-yl)-N′-benzylguanidine, and

pharmaceutically acceptable salts thereof.

In another embodiment, the compound of formula 1 is (2S,3S,4R)-N″-cyano-N-(6-amino-3,4-dihydro-3-hydroxy-2-methyl-2-dimethoxymethyl-2H-benzopyran-4-yl)-N′-benzylguanidine (DNB-001), and has the following exemplary structure:

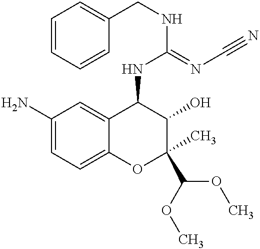

In some embodiments, the pharmaceutical formulation is suitable for application to an eye of a patient in need thereof For example, formulation may be provided as eyedrops, a gel or an ointment.

In some embodiments, the compound of formula 1 is provided as microparticles having an average diameter of about 0.5 to about 20 microns. The microparticles can be form by standard processes, such as by grinding or milling, for example wet milling or dry jet milling In some embodiments, the microparticles have an average diameter of about 1 to about 10 microns, about 1 to about 7 microns, about 1 to about 5 microns, or about 3 to about 5 microns. The microparticle size can also be characterized in that 75% of the particles have a diameter of less than 20 microns. In other embodiments, less than 75% of the microparticles have an average diameter of less than 15 microns, less than 10 microns, less than 5 microns, or less than 3 microns. In other embodiments, 99% of the particles have a diameter of less than 20 microns. In other embodiments, 99% of the microparticles have a diameter of less than 15 microns, less than 10 microns or less than 8 microns. In other embodiments, 99% of the microparticles have a diameter of less than 10 microns and 75% of the particles have a diameter of less than 5 microns.

In other embodiments, the compound of formula 1 is provided as nanoparticles. The nanoparticles may formed by standard processes, such as dry jet milling or wet milling. The nanoparticles also may be formed in the presence of other agents, such as surfactants and/or antifoaming agents, e.g., polyvinylpyrrolidone or polysorbate. The nanoparticles may have a diameter of less than about 0.5 microns or less than 0.3 microns. In other embodiments, the nanoparticles have an average diameter of about 0.05 microns to about 0.5 microns, 0.05 microns to about 0.3 microns or about 0.1 microns to about 0.1 microns. In another aspect, a formulation may include nano-particles having a diameter of less than 0.2 microns or less than 0.1 microns. These formulations are advantageous because they allow a stable suspension that can be sterilized with a standard 0.22 micron filter.

In a Pharmacokinetic (PK) Study, formulations including the nano-particles showed that the nano-particles provided three times the drug levels as micro-particles of the same active concentration. That is, formulations including the nano-particles of 1, 3, and 10% yielded the same drug levels in the aqueous humor as formulations including micro-particles of 3, 10, and 30%, respectively.

The formulation may contain other pharmaceutically acceptable ingredients, including but not limited to, solubilizing agents, buffers, isotonizers, preservatives, stabilizers, suspending agents, surfactants, chelating agents, thickening agents, and pH adjusting agents. For example, in some embodiments the formulation comprises:

about 1 to about 30% by weight of a compound of formula 1;

about 0.01 to about 30% by weight of a buffer;

about 0.01 to about 10% by weight of a chelator;

about 0.001 to about 10% by weight of a preservative;

about 0.1 to about 20% by weight of an isotonizer; and

about 0 to about 20% by weight of a solubilizing agent, suspending agent, surfactant, or thickening agent.

Examples of solubilizing agents include, for example, polysorbate 80, polyvinylpyrrolidone (such as Povidone), polyethylene glycol, propylene glycol, polyoxyethylene-hydrogenated castor oil 60, and polyoxyl stearate 40.

Examples of buffers that can be used in the formulations include boric acid or its salts such as borax, citric acid or its salts such as a sodium salt, tartaric acid or its salt such as a sodium salt, gluconic acid or its salt such as a sodium salt, acetic acid or its salts such as a sodium salt, phosphoric acid, monosodium hydrogen phosphate, disodium phosphate, disodium hydrogen phosphate, sodium dihydrogen phosphate, and amino acids.

Examples of isotonizers for use in the formulations include sorbitol, glucose, mannitol, glycerol, propylene glycol, sodium chloride, and potassium chloride.

Examples of preservatives for use in the formulations include p-hydroxy benzoate esters, benzalkonium chloride, benzethonium chloride, chlorobutanol, benzyl alcohol, sorbic acid or its salts, chlorhexidine gluconate, sodium dehydroacetate, cetylpyridinium chloride, and alkyldiaminoethylglycine hydrochloride.

Examples of stabilizers for use in the formulations include ascorbic acid, sodium edetate, cyclodextrin, condensed phosphoric acid or its salts, sulfite salts, citric acid or its salts.

Examples of suspending agents for use in the formulations include methylcellulose, hydroxypropylcellulose, polyvinylpyrrolidone, polyoxyethylenehydrogenated castor oil 60, polyoxyl stearate 40, polyethylene glycol, sodium carboxymethylcellulose, and poly vinylalcohol.

Examples of surfactants for use in the formulations include non-ionic surfactants, anionic surfactants, amphoteric surfactants and cationic surfactants. Examples of anionic surfactants include sodium lauryl sarcosinate, lauroyl-L-glutamic acid triethanolamine and sodium myristyl sarcosinate and the like. Examples of amphoteric surfactants for use in the formulations include lauryldimethylaminoacetic acid betaine, 2-alkyl-N-carboxymethyl-N-hydroxy-ethylimidazolinium betaine, alkyldiaminoglycine hydrochloride and the like. Examples of nonionic surfactants include polysorbate 80, polyoxyethylenehydrogenated castor oil 60, polyoxyl stearate 40, poloxamer 407, polaxamer 188, polyoxyethylene lauryl ether and the like. Examples of cationic surfactants include benzethonium chloride, benzalkonium chloride, cetylpyridinium chloride and the like.

Examples of chelating agents for use in the formulations include sodium edetate, sodium citrate, condensed phosphoric acid or its salts (condensed phosphates) and the like.

Examples of thickening agents for use in the formulations include methylcellulose, hydroxyethylcellulose, hydroxypropylcellulose, hydroxypropylmethylcellulose, sodium chondroitin sulfate, sodium carboxymethylcellulose, polyvinylpyrrolidone, polyvinylalcohol, polyethylene glycol and the like.

Examples of pH adjusting agents for use in the formulations include sodium hydroxide, potassium hydroxide, sodium carbonate, hydrochloric acid, phosphoric acid, citric acid, acetic acid and the like.

In some embodiments, the formulation comprises:

about 1 to about 30% by weight of a compound of formula 1;

about 0.01 to about 10% by weight of monobasic sodium phosphate monohydrate;

about 0.05 to about 20% by weight disodium phosphate heptahydrate;

about 0.01 to about 10% by weight of edetate disodium;

about 0.001 to about 10% by weight of benzalkonium chloride;

about 0.1 to about 20% by weight of sodium chloride;

about 0 to about 20% by weight of Polysorbate 80; and

water.

In other embodiments, the formulation comprises:

about 1 to about 15% by weight of a compound of formula 1;

about 0.01 to about 1% by weight of monobasic sodium phosphate monohydrate;

about 0.05 to about 2% by weight disodium phosphate heptahydrate;

about 0.01 to about 1% by weight of edetate disodium;

about 0.005 to about 0.1% by weight of benzalkonium chloride;

about 0.1 to about 2% by weight of sodium chloride;

about 0.01 to about 5% by weight of Polysorbate 80; and

water.

In other embodiments, the formulation comprises:

about 1 to about 10% by weight of a compound of formula 1;

about 0.01 to about 0.05% by weight of monobasic sodium phosphate monohydrate;

about 0.1 to about 0.5% by weight disodium phosphate heptahydrate;

about 0.01 to about 0.1% by weight of edetate disodium;

about 0.005 to about 0.05% by weight of benzalkonium chloride;

about 0.5 to about 1% by weight of sodium chloride;

about 0.01 to about 0.1% by weight of Polysorbate 80; and

water.

In other embodiments, the formulation comprises:

about 1 to about 30% by weight of a compound of formula 1;

about 0.01 to about 10% by weight of monobasic sodium phosphate monohydrate;

about 0.05 to about 20% by weight disodium phosphate heptahydrate;

about 0.01 to about 10% by weight of edetate disodium;

about 0.001 to about 10% by weight of benzalkonium chloride;

about 0.1 to about 20% by weight of sodium chloride;

about 0 to about 20% by weight of Povidone K29-32; and

water.

In other embodiments, the formulation comprises:

about 1 to about 15% by weight of a compound of formula 1;

about 0.01 to about 1% by weight of monobasic sodium phosphate monohydrate;

about 0.05 to about 2% by weight disodium phosphate heptahydrate;

about 0.01 to about 1% by weight of edetate disodium;

about 0.001 to about 0.5% by weight of benzalkonium chloride;

about 0.1 to about 5% by weight of sodium chloride;

about 0 to about 10% by weight of Povidone K29-32; and

water.

In other embodiments, the formulation comprises:

about 1 to about 10% by weight of a compound of formula 1;

about 0.01 to about 0.05% by weight of monobasic sodium phosphate monohydrate;

about 0.1 to about 0.5% by weight disodium phosphate heptahydrate;

about 0.01 to about 0.1% by weight of edetate disodium;

about 0.005 to about 0.05% by weight of benzalkonium chloride;

about 0.5 to about 1% by weight of sodium chloride;

about 1 to about 5% by weight of Povidone K29-32; and

water.

In other embodiments, the formulation comprises:

about 1 to about 10% by weight of (2S,3S,4R)-N″-cyano-N-(6-amino-3,4-dihydro-3-hydroxy-2-methyl-2-dimethoxymethyl-2H-benzopyran-4-yl)-N′-benzylguanidine;

about 0.01 to about 0.05% by weight monobasic sodium phosphate monohydrate;

about 0.1 to about 0.5% by weight disodium phosphate heptahydrate;

about 0.01 to 0.1% by weight edetate disodium;

about 0.01 to about 0.05% by weight benzalkonium chloride;

about 0.1 to about 1% by weight sodium, chloride;

about 0.01 to 0.1% by weight Polysorbate 80; and

water,

wherein the (2S,3S,4R)-N″-cyano-N-(6-amino-3,4-dihydro-3-hydroxy-2-methyl-2-dimethoxymethyl-2H-benzopyran-4-yl)-N′-benzylguanidine is provided as microparticles having an average diameter of about 1 to about 10 microns.

In other embodiments, the formulation comprises:

about 3% to about 10% by weight of (2S,3S,4R)-N″-cyano-N-(6-amino-3,4-dihydro-3-hydroxy-2-methyl-2-dimethoxymethyl-2H-benzopyran-4-yl)-N′-benzylguanidine;

about 0.02 to about 0.04% by weight monobasic sodium phosphate monohydrate;

about 0.1 to about 0.3% by weight disodium phosphate heptahydrate;

about 0.04 to 0.06% by weight edetate disodium;

about 0.01 to about 0.03% by weight benzalkonium chloride;

about 0.5 to about 1% by weight sodium, chloride;

about 0.03 to 0.5% by weight Polysorbate 80; and

water.

In other embodiments, the formulation comprises:

about 3% by weight of (2S,3S,4R)-N″-cyano-N-(6-amino-3,4-dihydro-3-hydroxy-2-methyl-2-dimethoxymethyl-2H-benzopyran-4-yl)-N′-benzylguanidine;

about 0.031% by weight monobasic sodium phosphate monohydrate;

about 0.203% by weight disodium phosphate heptahydrate;

about 0.049% by weight edetate disodium;

about 0.02% by weight benzalkonium chloride;

about 0.881% by weight sodium, chloride;

about 0.44% by weight Polysorbate 80; and

water,

wherein the (2S,3S,4R)-N″-cyano-N-(6-amino-3,4-dihydro-3-hydroxy-2-methyl-2-dimethoxymethyl-2H-benzopyran-4-yl)-N′-benzylguanidine is provided as microparticles having an average diameter of about 1 to about 10 microns.

In other embodiments, the formulation comprises:

about 10% by weight (2S,3S,4R)-N″-cyano-N-(6-amino-3,4-dihydro-3-hydroxy-2-methyl-2-dimethoxymethyl-2H-benzopyran-4-yl)-N′-benzylguanidine, or a pharmaceutically acceptable salt thereof;

about 0.029% by weight monobasic sodium phosphate monohydrate;

about 0.193% by weight disodium phosphate heptahydrate;

about 0.047% by weight edetate disodium;

about 0.019% by weight benzalkonium chloride;

about 0.837% by weight sodium, chloride;

about 0.042% by weight Polysorbate 80; and

and water.

In other embodiments, the formulation comprises:

about 1 to about 10% by weight of (2S,3S,4R)-N″-cyano-N-(6-amino-3,4-dihydro-3-hydroxy-2-methyl-2-dimethoxymethyl-2H-benzopyran-4-yl)-N′-benzylguanidine;

about 0.01 to about 0.05% by weight monobasic sodium phosphate monohydrate;

about 0.1 to about 0.5% by weight disodium phosphate heptahydrate;

about 0.01 to 0.1% by weight edetate disodium;

about 0.01 to about 0.05% by weight benzalkonium chloride;

about 0.1 to about 1% by weight sodium, chloride;

0 to about 5% by weight Polysorbate 80;

0 to about 5% by weight Povidone K29-32; and

water,

wherein the (2S,3S,4R)-N″-cyano-N-(6-amino-3,4-dihydro-3-hydroxy-2-methyl-2-dimethoxymethyl-2H-benzopyran-4-yl)-N′-benzylguanidine is provided as nanoparticles having an average diameter of less than 0.3 microns.

In other embodiments, the formulation comprises:

about 0.01 to about 0.05% by weight monobasic sodium phosphate monohydrate;

about 0.1 to about 0.5% by weight disodium phosphate heptahydrate;

about 0.01 to 0.1% by weight edetate disodium;

about 0.01 to about 0.05% by weight benzalkonium chloride;

about 0.1 to about 1% by weight sodium, chloride; and

about 1 to about 5% by weight Polysorbate 80.

In other embodiments, the formulation comprises:

about 0.01 to about 0.05% by weight monobasic sodium phosphate monohydrate;

about 0.1 to about 0.5% by weight disodium phosphate heptahydrate;

about 0.01 to 0.1% by weight edetate disodium;

about 0.01 to about 0.05% by weight benzalkonium chloride;

about 0.1 to about 1% by weight sodium, chloride; and

about 1 to about 5% by weight Povidone K29-32.

Another embodiment relates to a method of reducing intraocular pressure in a patient in need thereof comprising administering to an eye of the patient a pharmaceutically effective amount of any of the aforementioned pharmaceutical formulations. Another embodiment relates to a method of treating glaucoma in a patient in need thereof, comprising administering to the eye of a patient in need thereof a pharmaceutically effective amount of any of the aforementioned pharmaceutical formulations.

The formulation may be applied to the eye daily or several times each day, for example, one, two, three, four, five, six, seven, eight or more times daily. In one aspect, about 20 μl, 30 μl, 35 μl, 45 μl, or 50 μl, of the formulation may be applied to the eye at each dose. In another embodiment, about 20 μl to about 50 μl of the formulation may be administered at each dose. In another embodiment, about 35 μl to about 40 μl of the formulation may be administered at each dose.

In other embodiments, the formulation may be applied to the eye at an interval of less than daily, such as every two, three, four, five, six, seven, or more days.

The formulation may be applied to the eye daily over a length of time that may include days, weeks, months, or years. In one aspect, the formulation is applied to the eye for at least five consecutive days.

In some embodiments, intraocular pressure is reduced for at least 18 hours after application of the formulation to the eye. In another embodiment, the intraocular pressure is reduced for between about 2 to about 18 hours after application of the formulation to the eye.

In another some embodiments, the intraocular pressure is reduced by at least about 3, 4, or 5 mmHg. In another embodiment, intraocular pressure is reduced by about 5.6 mmHg. In yet another embodiment, the intraocular pressure is reduced by between about 3 to about 7 mmHg, 3 to about 6 mmHg, or about 4.1 to about 5.6 mmHg.

The compounds of formula 1 may be prepared by the following illustrative scheme 1:

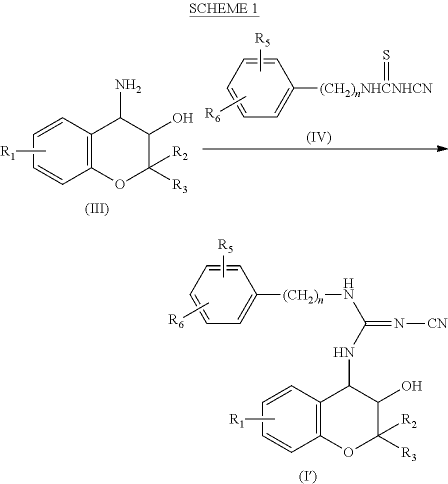

wherein R₁, R₂, R₃, R₄, R₅, R₆ and n are each defined as above.

The benzopyranyl guanidine derivatives of formula 1 may also be prepared by the following scheme 2:

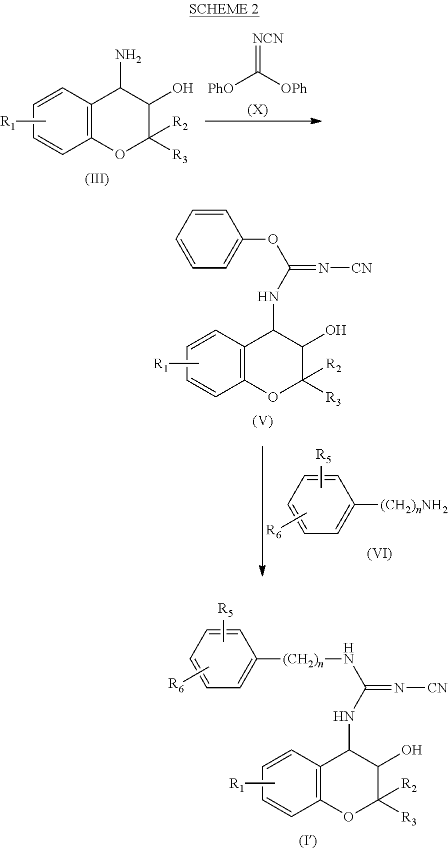

wherein R¹, R² R³, R⁴, R⁵, R⁶ and n are each defined as above.

The compounds of formula 1 may also be prepared by using the compound (I′) prepared in scheme 1 or scheme 2, as shown below in scheme 3:

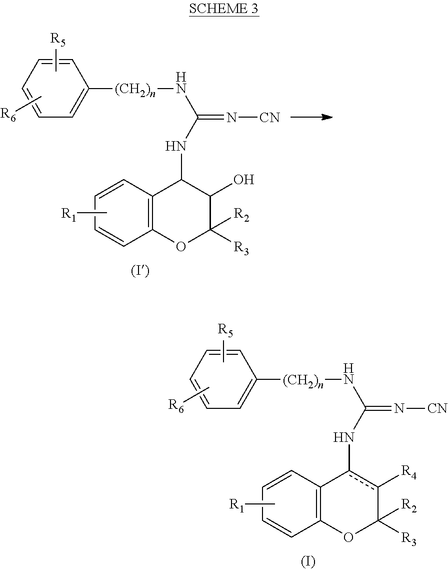

wherein R¹, R², R³, R⁴, R⁵, R⁶ and n are each defined as above.

The substituents of R¹, R², R³, R⁴, R⁵, and R⁶ can be modified or a 3,4-double bond can be formed via the reaction represented by the above scheme 3.

The compounds of formula 1 may also be prepared separately as an optically active isomer by using the corresponding optical isomer as a starting material.

In case of using a racemic mixture as a starting material, the compounds of formula 1 can be prepared as a racemic mixture, and then the racemic mixture can be separated into each optical isomer. The optical isomers can be separated, for example, by chiral column chromatography or recrystallization by methods know in the art.

The compounds of formula 1 can be synthesized using the reactions and techniques described herein below. The reactions can be performed in a solvent appropriate to the reagents and materials employed and suitable for the transformation being effected.

I. Preparation of Starting Materials

Aminoalcohol compounds (III), which can be used as a starting material in scheme I or scheme 2, can be prepared by the reaction represented by the following scheme 4,

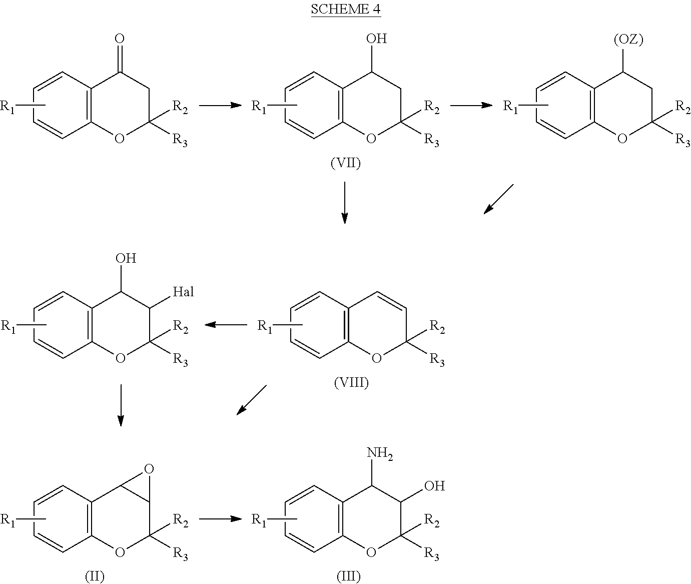

wherein R₁, R₂ and R₃ are each defined as above, (OZ) represents a leaving group, and Hal represents a halogen atom.

The method for the preparation of the epoxide compound (II) represented by the above scheme 4 is described in detail in U.S. Pat. No. 5,236,935 and KR Pat. No. 096,546, which are incorporated herein by reference in their entireties. The epoxide compound (II) can be prepared from propazylether derivatives as described in J. Med. Chem. 26, 1582 (1983).

(1) Preparation of Olefin Compounds (VIII)

Olefin compounds (VIII) exist as enantiomers (VIII₁ and VIII₂) such as formula 4,

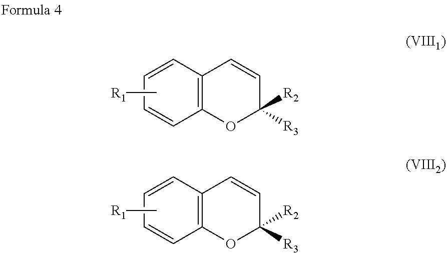

wherein R₁, R₂ and R₃ are each defined as above.

Olefin compounds (VIII) can be obtained separately as an optically active olefin compound (VIII₁) and olefin compound (VIII₂) of formula 4, respectively. The olefin compound (VIII) can be prepared by, for example, the method disclosed in KR Pat. Appln. No. 96-7399.

Scheme 5 below shows the detailed process for the preparation of the olefin compound (VIII) from an alcohol compound (VII):

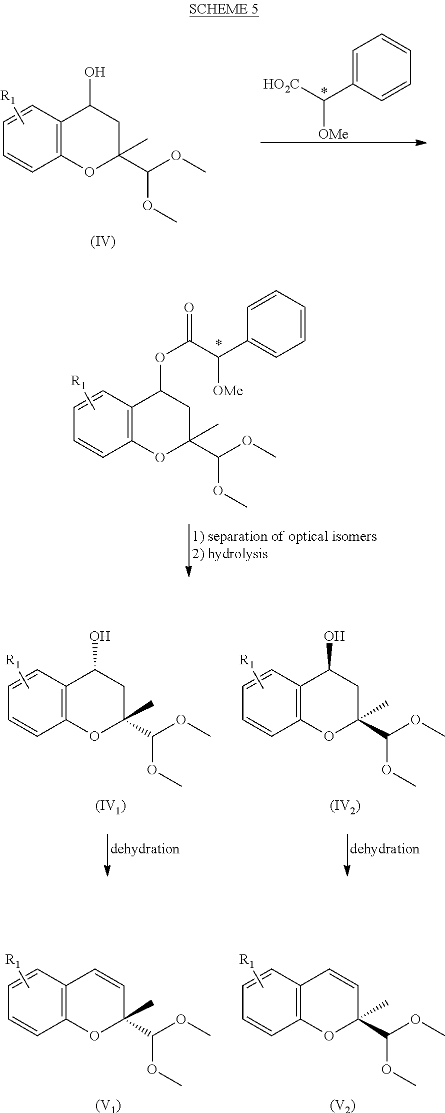

wherein R¹, R² and R³ are each defined as above.

(2) Preparation of Epoxide Compounds (II)

Epoxide compounds (II₁) and epoxide compounds (II₂) can be prepared from the compound (VIM), and epoxide compounds (II₃) and epoxide compounds (II₄) can be prepared from the compound (VIII₂), as represented by the following scheme 6, by using the compound (VIII₁) and the compound (VIII₂) prepared in scheme 5 as a starting material, respectively,

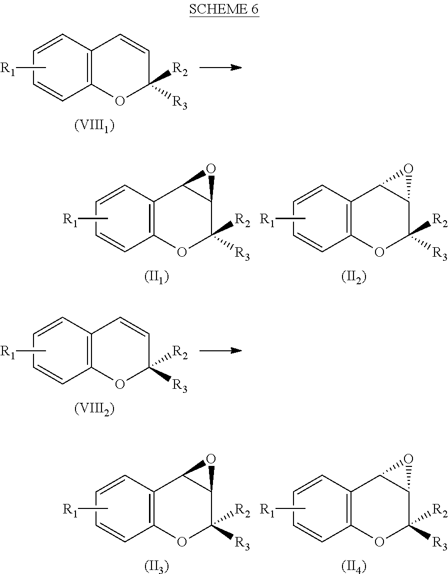

wherein R¹, R² and R³ are each defined as above.

The epoxide compounds (II₁) and (II₂) can be separated to each optical isomer, and the separated epoxide compounds or the mixture thereof can be used to prepare aminoalcohol compounds as described below. The epoxide compounds (II₃) and (II₄) can be separated, and the separated epoxide compounds or the mixture thereof can be used to prepare aminoalcohol compounds as described below.

Epoxide compounds (II₁) and (II₂) and epoxide compounds (II₃) and (II₄) can be prepared, for example, from olefin compounds (VIII₁) and (VIII₂), respectively, by the preparation method disclosed in U.S. Pat. No. 5,236,935 and KR Pat. No. 096,546.

Optical isomers (II₁), (II₂), (II₃) and (II₄) of epoxide compounds can be prepared, for example, from olefin compounds (VIII₁) or (VIII₃), by using Mn(III) salen epoxidation catalysts as described in E. N. Jacobsen et al., Tetrahedron Lett., 38, 5055 (1991). Epoxide compounds (II₁) can be prepared from olefin compounds (VIII₁) and epoxide compounds (II₃) from the olefin compounds (VIII₂) using, for example, (R,R)—Mn(III) salen catalyst, e. Epoxide compounds (II₂) can be prepared from the olefin compounds (VIII₁) and epoxide compounds (II₄) from the olefin compounds (VIII₂) using, for example, (S,S)—Mn(III) salen catalyst. This epoxidation reaction can be performed, for example, in a mixture of methylene chloride and water by using NaOCl as an oxidizing agent.

(3) Preparation of Aminoalcohol Compounds (III)

Aminoalcohol compounds (III) can be prepared by the reaction of the epoxide compound (II) with ammonia gas (NH₃) or ammonium hydroxide (NH₄ OH) in the presence of a suitable solvent, in the above scheme 4. Suitable solvents include alcohols (e.g., methanol, ethanol, and isopropanol). The reaction temperature may range from about 5° C. to the boiling point of the solvent employed.

Aminoalcohol compounds (III₁), (III₂), (III₃) and (III₄) can be obtained using each epoxide compound (II₁), (II₂), (II₃) and (II₄) as a starting material, respectively. A mixture of aminoalcohol compounds (III₁), and (III₂) can be obtained using a mixture of epoxy compound (II₁) and (II₂) as a starting material. A mixture of aminoalcohol compounds (III₃) and (III₄) can be obtained using a mixture of epoxide compound (II₃) and (II₄) as a starting material,

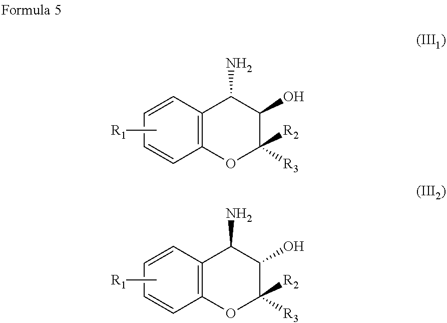

wherein R¹, R² and R³ are each defined as above, or

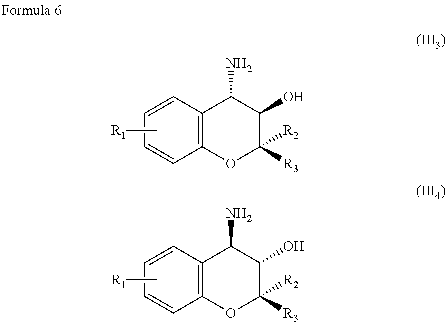

wherein R¹, R² and R³ are each defined as above.

II. Preparation Method I

One method for the preparation of the benzopyranyl guanidine derivatives of formula 1 comprises the step of reacting an aminoalcohol compound (III) and a thiourea compound (N) in the presence of a suitable condensing agent and a suitable solvent. The compound (I′), which is a compound of formula 1 wherein R₄═H, may be is prepared by this reaction.

Examples of such condensing agents include carbodiimide-type condensing agents such as 1-[3-(dimethylamino)propyl]-3-ethylcarbodiimide hydrochloride, and N,N′-dicyclohexylcarbodiimide, etc. Water-soluble carbodiimide-type condensing agents may also be employed.

In one aspect, one to three equivalents of the condensing agent can be used in place of aminoalcohol compound (III). One to two equivalents of thiourea compound (IV) can be used in place of the aminoalcohol compound (III). Solvents, including, but not limited to, methylene chloride, chloroform, dimethylformamide, dimethylsulfoxide, tetrahydrofuran, 1,2-dichloroethane, and dioxane can be used. Reaction temperature may range from about 5° C. to about 40° C.

A product with the same configuration to that of the starting material can be obtained using each stereoisomer of the aminoalcohol compound (III) as a starting material. For example, compounds (I₁), (I₂), (I₃) and (I₄) of formula 1 can be prepared from aminoalcohol compounds (III₁), (III₂), (III₃) and (III₄), respectively. A mixture of compounds (I₁) and (I₂) can be prepared using a mixture of aminoalcohol compounds (III₁) and (III₂) as a starting material. A mixture of compounds (I₃) and (I₄) can be prepared using a mixture of aminoalcohol compounds (III₃) and (III₄) as a starting material. A mixture of the benzopyranyl guanidine derivative of formula 1 can be separated to prepare separated optical isomers. In one aspect, the optical isomers can be separated by column chromatography or recrystallization using known techniques.

The thiourea compound (IV) used in the above reaction can be prepared by the reaction of an isocyanate compound (IX) with sodium cyanamide (NaHNCN) in ethanol as represented by the following scheme 7:

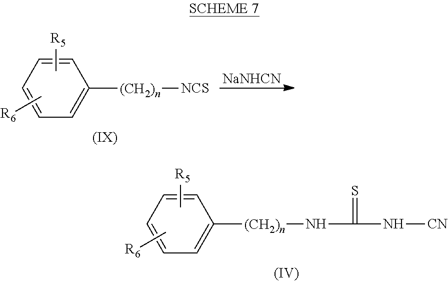

wherein R⁵, R⁶ and n are each defined as above.

III. Preparation Method II

Another method for the preparation of the benzopyranyl guanidine derivatives of formula 1 comprises

1) reacting an aminoalcohol compound (III) with diphenyl cyanocarbonimidate (X) in the presence of a base to prepare the compound (V) (step 1); and

2) reacting the compound (V) with an appropriate amine compound (VI) in a suitable solvent to prepare the compound (I′) (step 2).

The compound (I′) which is a compound of formula 1 with R₄═CH, can be prepared by this reaction.

Various inorganic and organic bases can be employed in step 1. Inorganic bases that can be used include, for example, CaCO₃, NaOH, KOH, Na₂O₃, and NaHCO₃. Organic bases including metal salts of alcohols (e.g., sodium methoxide (CH₃ONa), sodium ethoxide (CH₃CH₂ONa) etc.; sodium acetate (CH₃COONa)); metal salts of ammonia; bicyclic amines (e.g., 1,8-diazabicyclo[5.4.0]undec-7-ene (DBU), 1,5-diazabicyclo[4.3.0]non-5-ene (DBN), etc.; triethylainine; N,N-diisoprocylethylamine; pyridine; lutidine; N,N-dimethylaniline; 4-(dimethylamino)-pyridine; 1,4-diazabicyclo[2.2.2]octane (DABCO)) can also be used in step 1. Exemplary tertiary amines (e.g.,triethylamine, N,N-diisopropylethylamine, pyridine, 1,8-diazabicyclo[5.4.0]undec-6-ene, and 4-(dimethylamino)pyridine) can be used in step 1.

In one aspect, one to three equivalents of a base can be used in place of the aminoalcohol compound (III). For example, diphenyl cyanocarbonimidate (X) can be used in place of the aminoalcohol compound (III).

In another aspect, solvents such as ethanol, isopropanol, etc., dimethylformamide (DMF), dimethylsulfoxide (DMSO), and chloroform can be used in step 1.

In yet another aspect, reaction temperature can be maintained from about 5° C. to the boiling point of the solvent employed in step 1.

In step 2, one to five equivalents of the amine compound (VI) can be used in place of the aminoalcohol compound (III).

Examples of the reaction solvent include alcohols such as ethanol, isopropanol, etc., dimethylformamide, dimethylsulfoxide, chloroform, methylene chloride, and tetrahydrofuran (THF), etc.

Reaction temperature can be maintained from about 5° C. to the boiling point of the solvent employed in step 2.

In addition, the reaction of step 2 can be carried out in the presence of a base, such as the bases described above.

Product (I′) with the same configuration of the starting material can be obtained by using each optical isomer of aminoalcohol compound (III) as a starting material. A mixture of compounds (I₁) and (I₂) can be obtained using a mixture of aminoalcohol compounds (II₁) and (III₂) as a starting material. A mixture of compounds (I₃) and (I₄) can be obtained using a mixture of aminoalcohol compounds (III₃) and (III₄) as a starting material. Separated optical isomers of compounds (I′) can be prepared from a mixture of optical isomers and separated by, for example, column chromatography or recrystallization.

IV. Preparation of Compounds (I) from Compounds (I′)

In another aspect, the substituents R¹, R², R₃, R₄, R⁵ or R⁶ can be modified to other functional groups, and a double bond can be formed at 3,4-position by the reaction of scheme 3. In this reaction, the compounds (I′), prepared by the above scheme 1 or scheme 2, can be used as a starting material.

Starting materials, reactants, and the reaction conditions can be determined according to the structure of product (e.g., the substituents R¹, R², R³, R⁴, R⁵ or R⁶ and whether there is a bond at 3,4-position). Therefore, one of skill in the art using the present disclosure can readily determine the reaction types, reactants and reaction condition by which it is possible to prepare the benzopyranyl guanidine derivatives of formula 1.

(1) Introduction of Acetoxy Group at R⁴

An acetoxy group can be introduced at R⁴ by reacting the compound (I′) with acetic anhydride in suitable solvent in the presence of a suitable catalyst as represented in the below scheme 8,

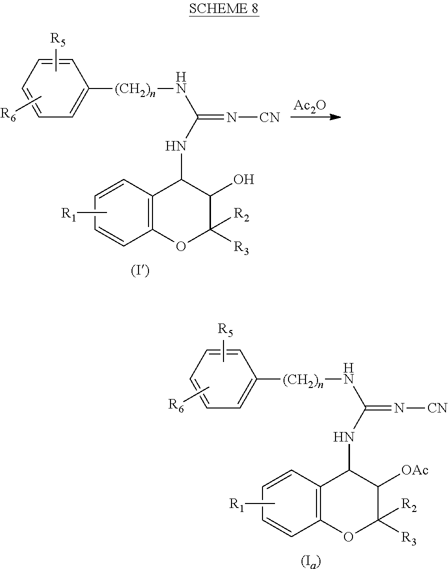

wherein R¹, R², R³, R⁴, R⁵, R⁶ and n are each defined as above.

Exemplary bases for use in this scheme are described above. Triethylamine, pyridine or N,N-diisopropylethylamine, for example, may be used.

The catalyst used in the scheme may be 4-(dimethylamino)pyridine. One to three equivalents of the base can be used in place of compound (I′). In addition, 0.05-0.5 equivalents of the catalyst can be used in place of compound (I′).

Solvents suitable for use in scheme 8 include, but are not limited to, methylene chloride, chloroform, tetrahydrofuran, and acetonitrile. The reaction temperature can be from about 0 to about 40° C.

(2) Introduction of Double Bond at 3,4-Position

R₄ is substituted with hydrogen atom and a double bond is formed at 3,4-position by reacting the acetate compound (Iₐ) prepared in the above scheme 8 with suitable base in suitable solvent as represented in the below scheme 9,

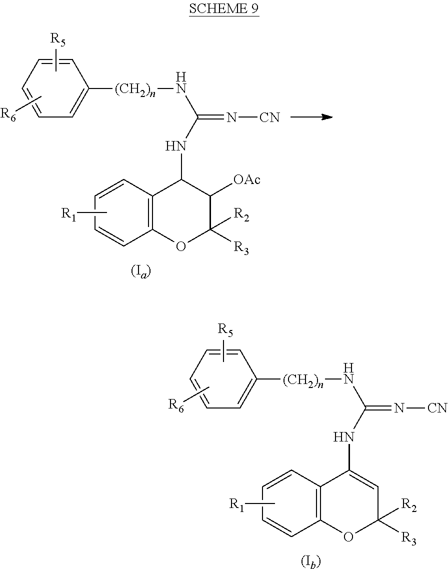

wherein R¹, R², R³, R⁴, R⁵, R⁶ and n are each defined as above.

The bases described above can be used in scheme 9. Other bases that may be used include, but are not limited to, 1,8-diazabicyclo[5.4.0]undec-7-ene, 1,5-diazabicyclo[4.3.0]non-5-ene or 1,4-diazabicyclo[2.2.2]octane. One to three equivalents of the base may be used in place of compound (Iₐ).

Solvents that may be used include, but are not limited to, toluene, benzene, xylene, dioxane, etc. The reaction temperature may be from about 5° C. to the boiling point of the solvent.

(3) Introduction of NH₃ Group at R₁ p The compound (I_{d}) of formula 1 whose R₁ is NH₂ can be prepared by the reduction of the compound (L) with R₁═NO₂ as represented in the below scheme 10,

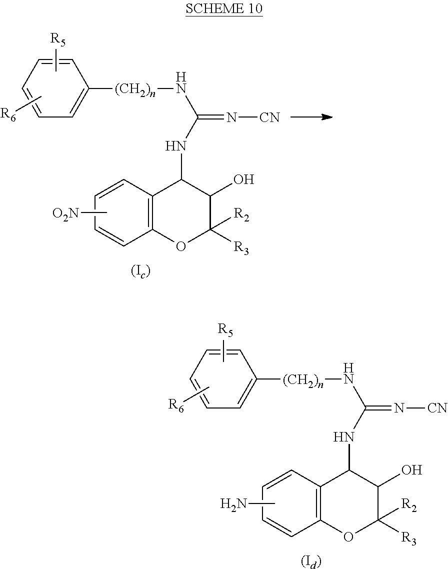

wherein R², R³, R⁵, R⁶ and n are each defined as above.

The NO₂ group can be reduced to NH₂ group by hydrogenation using metal catalysts such as platinum, palladium, palladium on carbon (Pd/C), Paney-nickel, etc. in a suitable solvent. The solvent may be alcohols such as methanol, ethanol, etc., and ethyl acetate.

In addition, the reduction of NO₂ group to NH₂ group can be carried out by using a reducing agent such as NaBH₄ in the presence of CuSO₄, Cu(OAc)₂, CoCl, SnCl₂ or NiCl₂. The solvent may be, for example, a mixture of water and methanol and reaction temperature may be room temperature.

(4) Introduction of NH(C═O)R^{a} at R₁

The compound of formula 1 with R¹═NH(C═O)R^{a} can be prepared by the reaction of the compound (I_{d}) prepared in the above scheme 10 with acetylchloride or acid anhydride in the presence of a base.

Suitable bases include the bases described above. Other suitable bases include, but are not limited to, triethylamine, N,N-diisopropylethylamine, pyridine or 4-(dimethylamino)pyridine. Preferred solvents are methylene chloride, chloroform, dimethylsulfoxide, dimethylformamide, tetrahydrofuran, and dioxane.

(5) Introduction of —NHS(O)ₘR^{a} at R₁

The benzopyranyl guanidine derivatives of formula 1 with R₁═—NHS(O)ₘR^{a} can be prepared by the reaction of the compound (Id) as prepared in scheme 10 with alkylsulfonyl chloride or arylsulfonyl chloride in the presence of a base.

Suitable bases include the bases described above. Other suitable bases include, but are not limited to, triethylamine, N,N-diisopropylethylamine, pyridine and 4-(dimethylamino)pyridine. Preferred solvents are methylene chloride, chloroform, dimethylsulfoxide, dimethylformamide, tetrahydrofuran, and dioxane.

### Preparation of (2S,3R,4S)-N″-cyano-N-(6-amino-3,4-dihydro-3-hydroxy-2-methyl-2-dimethoxymethyl-2H-benzopyran-4-yl)-N′-benzylguanidine

Step 1: 100 ml of water was added to a solution of 5 g (19 mmol) of (2R)-6-nitro-2-methyl-2-dimethoxymethyl-2H-1-benzopyran in 100 ml of acetone. 5.6 g (66 mmol) of sodium hydrogen carbonate was added to the mixture and the mixture was stirred for 10 min. 11.6 g (19 mmol) of oxone was added to the mixture and the mixture was strongly stirred. Sodium hydrogencarbonate and oxone were added to the mixture an additional three times at 15 minute intervals. The reaction mixture was filtered, acetone was removed under reduced pressure, and the residue was extracted two times using 100 ml ethyl acetate each time. The organic layer was dried over anhydrous magnesium sulfate, filtered, concentrated under reduced pressure and purified by silica gel column chromatography (n-hexane:ethyl acetate=4:1) to afford 5.1 g (yield: 97%) of the desired compound, a white solid as a racemic mixture.

Step 2: 5.1 g of the epoxide compound prepared in Step 1 was dissolved in 100 ml of the saturated ammonia ethanol and the reaction mixture was stirred for 7 days at room temperature. The solvent was removed and the residue was purified by silica gel column chromatography (n-hexane:ethyl acetate=1:4) to afford 4.4 g (yield: 80%) of the desired compound as a racemic mixture.

Step 3: 1 L of water was added to a solution of 508 mg of N-cyano-N′-benzylthiourea sodium salt and 500 mg of the aminoalcohol compound prepared in Step 2 in 1 L of acetone. 84 g (0.99 mol) of sodium hydrogen carbonate was added to the mixture and the mixture was stirred for 10 min. 174 g (0.28 mol) of oxone was added to the mixture and the mixture was strongly stirred. Sodium hydrogencarbonate and oxone were added to the mixture an additional three times at 15 minute intervals. The reaction mixture was filtered, acetone was removed under reduced pressure and the residue was extracted twice with 500 ml ethyl acetate each time. The organic layer was dried over anhydrous magnesium sulfate, filtered, concentrated and purified by silica gel column chromatography (n-hexane:ethyl acetate=4:1).

Step 4: The epoxide compound prepared in the Step 3 was dissolved in 250 ml of the saturated ammonia ethanol and the reaction mixture was stirred for 7 days at room temperature. The solvent was removed and the residue was purified by silica gel chromatography (n-hexane:ethyl acetate=1:1) to afford 170 mg (yield: 22%) of the compound of (2S,3R,4S) stereochemistry. Alternatively, it would have been possible to proceed to step 5 below using a compound of (2S,3S,4R) stereochemistry.

Step 5: 0.5 ml (0.2 mmol, 0.5 eq) of 0.4M aqueous Cu(OAc)₂ solution was added to a solution of 884 mg (1.94 mmol) of the compound prepared in Step 4 dissolved in 5 ml of methanol. 155 mg (4.1 mmol, 10 eq) of sodium borohydride was added to this mixture slowly for 30 min. The reaction mixture was stirred for 1 hour at room temperature, extracted with 10 ml of ethyl acetate, and filtered to remove a precipitated black solid. The filtered solution was extracted with 30 ml of ethyl acetate after adding 10 ml of the saturated aqueous NaHCO₃ solution. The organic layer was washed with brine, dried over anhydrous magnesium sulfate, filtered and concentrated under reduced pressure. The residue was purified by silica gel chromatography (n-hexane:ethyl acetate=1:3) to afford 313 mg (yield: 38%) of the desired compound.

¹H NMR (CDCl₃, 500 MHz) δ 1.41 (s, 3H), 1.75 (br s, 1H), 3.39 (s, 3H), 3.45 (s, 3H), 3.46 (d, 1H), 3.72 (d, 1H), 4.40 (s, 1H), 4.46 (d, 2H), 4.78 (d, 1H), 5.22 (m, 1H), 6.41 (m, 1H), 6.50 (m, 1H), 6.59 (d, 1H), 6.73 (m, 1H), 7.30-7.37 (m, 4H)

In addition to the cardioprotective and neuroprotective activity from ischemia-reperfusion and hypoxic damage, the benzopyranyl guanidine derivatives represented by formula 1 can be used in a method to reduce intraocular pressure in an eye, for example, a human eye affected by glaucoma, by topical application to the eye.

### Exemplification

Micronized DNB-001 formulations: DNB-001 micronization was performed by Micron Technologies using a jet milling process with mill JAM-4-001 at a starting pressure of 100 psi and a feed rate of about 600 g/hour. The carrier was prepared by mixing monobasic sodium phosphate monohydrate, disodium phosphate heptahydrate, edetate disodium dehydrate, benzalkonium chloride, sodium chloride, and either povidone K29-32 or polysorbate 80. The micronized DNB-001 was then mixed with the carrier to provide the final formulations as described in tables 1 and 2.

TABLE 1

| | | 1 | 2 |
|---|---|---|---|
| Ingredient | % w/w | % w/w | % w/w |
| Monobasic Sodium Phosphate, | 0.0312 | 0.030 | 0.028 |
| monohydrate | | | |
| Disodium phosphate, heptahydrate | 0.2075 | 0.201 | 0.187 |
| Edetate Disodium (EDTA, disodium | 0.05 | 0.049 | 0.045 |
| dehydrate) | | | |
| Benzalkonium chloride | 0.02 | 0.019 | 0.018 |
| Sodium chloride | 0.72 | 0.698 | 0.648 |
| Povidone K29-32 | 1.8 | 1.746 | 1.620 |
| DNB-001 | 0-10 | 3 | 10 |
| Purified Water qs | 100 | 100 | 100 |

TABLE 2

| | | 3 | 4 | 5 | 6 |
|---|---|---|---|---|---|
| Ingredient | % w/w | % w/w | % w/w | % w/w | % w/w |
| Monobasic Sodium Phosphate, monohydrate | 0.0312 | 0.031 | 0.029 | 0.027 | 0.022 |
| Disodium phosphate, heptahydrate | 0.2075 | 0.203 | 0.193 | 0.178 | 0.145 |
| Edetate Disodium (EDTA, disodium dehydrate) | 0.05 | 0.049 | 0.047 | 0.043 | 0.035 |
| Benzalkonium chloride | 0.02 | 0.020 | 0.019 | 0.017 | 0.014 |
| Sodium chloride | 0.9 | 0.881 | 0.837 | 0.774 | 0.630 |
| Polysorbate 80 | 0.045 | 0.044 | 0.042 | 0.039 | 0.032 |
| DNB-001 | 0-30 | 3 | 10 | 20 | 30 |
| Purified Water qs | 100 | 100 | 100 | 100 | 100 |

Nanoparticle DNB-001 formulations: Nanoparticles were prepared by Netzsch using a wet milling process in water with a MicroCer mill with 0.3 mm YTZ beads, a mill screen of 0.1 mm, a 2500 rpm starting agitator speed and a pump speed of 100 ml/minute. For the Povidone containing formulations, 37.5 g DNB-001 was fed to a 212.5 g DI water/5 g povidone solution circulating in the mill over a five minute period. After 30 minutes of milling the agitator speed was increased to 3500 rpm. After 90 minutes of milling 3 g of Povidone was added, bringing the total up to 8 g and the agitator speed was increased to 4000 rpm. The bead and screen were changed at 135 minutes to 0.1 mm YTZ beads and a 0.05 mm screen. The agitator speed was set to 3500 rpm after the bead change (135 minutes) and then increased to 4000 rpm at 180 minutes. The viscosity was reduced immediately from a toothpaste consistency to water consistency. The viscosity built up again at 120 minutes and at 155 minutes of milling 3 g of Povidone was added, to a total of 11 g. The viscosity decreased immediately, but then it went up again after 175 minutes of milling. At 205 minutes, the sample was pumped out of the mill.

The carrier was prepared by mixing the remaining ingredients. The nanoparticles of DNB-001 were then mixed with the carrier to provide the final formulation, as described in Table 3.

Nanoparticle DNB-001 formulations: Nanoparticles were prepared by Netzsch using a wet milling process in water with a MicroCer mill with 0.3 mm YTZ beads, a mill screen of 0.1 mm, 2500 rpm starting agitator speed and a pump speed of 100 ml/minute. For the Polysorbate containing formulation, 37.5 g DNB-001 was fed to a 212.5 g DI water/5 g Polysorbate solution circulating in the mill over a five minute period. After 30 minutes of milling the agitator speed was increased to 3500 rpm. After 90 minutes of milling 3 g of Polysorbate was added, bringing the total up to 8 g and the agitator speed was increased to 4000 rpm. The bead and screen were changed at 135 minutes to 0.1 mm YTZ beads and a 0.05 mm screen. The agitator speed was set to 3500 rpm after the bead change (135 minutes) and then increased to 4000 rpm at 180 minutes. 2 g Polysorbate was added at 180 minutes, bringing the total to 10 grams. At 205 minutes, the sample was pumped out of the mill.

The carrier was prepared by mixing the remaining ingredients. The nanoparticles of DNB-001 were then mixed with the carrier to provide the final formulation, as described in Table 3.

TABLE 3

| | | 7 | 8 | 9 | 10 |
|---|---|---|---|---|---|
| Ingredient | % w/w | % w/w | % w/w | % w/w | % w/w |
| Monobasic Sodium Phosphate, monohydrate | 0.0312 | 0.031 | 0.030 | 0.028 | 0.031 |
| Disodium phosphate, heptahydrate | 0.2075 | 0.205 | 0.201 | 0.187 | 0.205 |
| Edetate Disodium (EDTA, disodium dehydrate) | 0.05 | 0.050 | 0.049 | 0.045 | 0.050 |
| Benzalkonium chloride | 0.02 | 0.020 | 0.019 | 0.018 | 0.020 |
| Sodium chloride | 0.9 | 0.891 | 0.873 | 0.810 | 0.891 |
| Povidone K29-32 | 4.2 | 4.200 | 4.200 | 4.200 | 0.000 |
| Polysorbate 80 | 3.9 | 0.000 | 0.000 | 0.000 | 3.900 |
| DNB-001 | 0-30 | 1 | 3 | 10 | 1 |
| Purified Water qs | 100 | 100 | 100 | 100 | 100 |

In vivo study: A multiple-dose study was performed in glaucomatous monkey eyes. Eight eyes topically received concentrations of either 3% or 10% by weight of a formulation containing DNB-001, as described in Table 1, formulae 1 and 2. Intraocular pressure was measured hourly for 6 hours on each study day. Following one untreated baseline day and one vehicle-treated day, 25 μl of either the 3% or 10% formulation was applied topically to the glaucomatous eyes twice daily for 5 consecutive days.

The 3% formulation reduced the intraocular pressure (p<0.05) at 2 hrs and 4 hrs after the morning dosing on day 3 and 2 hrs and 3 hrs after the morning dosing on day 5. The maximum reduction in intraocular pressure was 4.1±0.8 (mean±SEM) mmHg (14%) 2 hrs after the morning dosing on day 5.

The 10% formulation reduced (p<0.05) intraocular pressure beginning 1 hr after the morning dose on day 3 and intraocular pressure remained decreased through 6 hrs post-dosing. The maximum intraocular pressure reduction on day 3 was 4.4±0.5 mmHg (15%) at 2 hrs after the morning dosing. On treatment day 5, a reduction (p<0.05) in intraocular pressure was observed from 0 hr to 6 hrs with the maximum reduction in intraocular pressure of 5.6±1.0 mmHg (19%) at 2 hrs post-dosing. The longest duration of intraocular pressure reduction was observed after repeated dosing with the 10% formulation. Compared with the 3% formulation, the 10% formulation had a longer duration of action (18 vs. 2 hrs) and caused a greater (p<0.05) reduction in intraocular pressure. Ocular side effects were not observed. The topically applied 3% and 10% formulations reduced intraocular pressure in glaucomatous monkey eyes in a dose-dependent manner.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### Equivalents

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed.

We claim:

1. A topical pharmaceutical formulation, comprising:
a compound of formula 1: 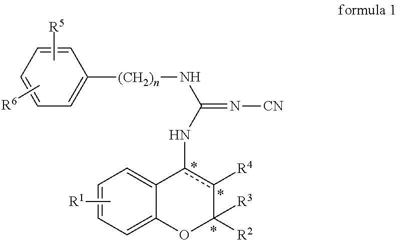
wherein:
R¹ represents H, halogen, CF₃, NO₂, CN, OR^{a}, O(C═O)R^{a}, COOR^{a}, NH₂, NHS(O)ₘR^{a}, NH(C═O)R^{a} or S(O)ₘR^{a}; R^{a} represents straight or branched alkyl group of C₁-C₄ or aryl group; and m is an integer of 0-2,
R² represents straight or branched alkyl group of C₁-C₄,
R³ represents CH₂ OR^{a}, 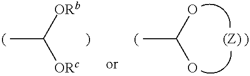
R^{a} is defined as above; R^{b} and R^{c} are independent of each other and represent a straight or branched alkyl group of C₁-C₄, respectively; and Z represents a straight or branched alkyl group of C₁-C₅,
R⁴ represents OH, H, halogen, ONO₂ or O(C═O)R^{a}; and R^{a} is defined as above;
R⁵ and R⁶ are independent of each other and represent H, halogen, straight or branched alkyl group of C₁-C₃, OR^{a}, CX₃, NO₂, CO₂Rₐ, —(C═O)Rₐ or SO₃R^{a}; R^{a} is defined as above; and X represents halogen,
n is an integer of 0-2,
and * represents the chiral center;
wherein the compound of formula 1 is provided as microparticles or nanoparticles having an average diameter of about 0.1 micron to about 20 microns; and
a pharmaceutically acceptable carrier comprising a solubilizing agent; a surfactant, a buffer, a chelator, a suspending agent, an isotonizer, a preservative, a stabilizing agent, a thickening agent, a pH adjusting agent, or mixtures thereof.

2. The formulation of claim 1, wherein the formulation is provided as eyedrops, a gel or an ointment.

3. The formulation of claim 1, wherein
R¹ represents NO₂, CN, NH₂ or S(O)ₘR^{a}; R^{a} represents a straight or branched alkyl group of C₁-C₂, or an aryl group; and m is an integer of 0-2,
R² represents CH₃,
R³ represents 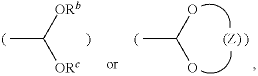
R^{b} and R^{c} are independent of each other and represent straight or branched alkyl groups of C₁-C₃, respectively; and Z represents straight or branched alkyl group of C₁-C₅,
R⁴ represents OH, H or O(C═O)R^{a}; and R^{a} represents a straight or branched alkyl group of C₁-C₃;
R⁵ and R⁶ are independent of each other and represent H, halogen, straight or branched alkyl groups of C₁-C₃, OR^{a}, CX₃ or NO₂; R^{a} represents a straight or branched alkyl group of C₁-C₃; and X represents halogen, and
n is an integer of 0-2.

4. The formulation of claim 1, wherein the compound of formula 1 is selected from the group consisting of:
1) (2R,3R,4S)-N″-cyano-N-(6-nitro-3,4-dihydro-3-hydroxy-2-methyl-2-dimethoxymethyl-2H-benzopyran-4yl)-N′-(4-chlorophenyl)guanidine;
2) (2R,3S,4R)-N″-cyano-N-(6-nitro-3,4-dihydro-3-hydroxy-2-methyl-2-dimethoxymethyl-2H-benzopyran-4-yl)-N′-(4-chlorophenyl)guanidine;
3) (2R,3R,4S)-N″-cyano-N-(6-nitro-3,4-dihydro-3-hydroxy-2-methyl-2-dimethoxymethyl-2H-benzopyran-4-yl)-N′-(3-chlorophenyl)guanidine;
4) (2R,3S,4R)-N″-cyano-N-(6-nitro-3,4-dihydro-3-hydroxy-2-methyl-2-dimethoxymethyl-2H-benzopyran-4-yl)-N′-(3-chlorophenyl)guanidine;
5) (2R,3R,4S)-N″-cyano-N-(6-nitro-3,4-dihydro-3-hydroxy-2-methyl-2-dimethoxymethyl-2H-benzopyran-4-yl)-N′-(4-nitrophenyl)guanidine;
6) (2R,3R,4S)-N″-cyano-N-(6-nitro-3,4-dihydro-3-hydroxy-2-methyl-2-dimethoxymethyl-2H-benzopyran-4-yl)-N′-(3-trifluoromethylphenyl)guanidine;
7) (2R,3S,4R)-N″-cyano-N-(6-nitro-3,4-dihydro-3-hydroxy-2-methyl-2-dimethoxymethyl-2H-benzopyran-4-yl)-N′-(3-trifluoromethylphenyl)guanidine;
8) (2R,3R,4S)-N″-cyano-N-(6-nitro-3,4-dihydro-3-hydroxy-2-methyl-2-dimethoxymethyl-2H-benzopyran-4-yl)-N′-(4-methoxyphenyl)guanidine;
9) (2R,3S,4R)-N″-cyano-N″-6-nitro-3,4-dihydro-3-hydroxy-2-methyl-2-dimethoxymethyl-2H-benzopyran-4yl)-N′-(4-methoxyphenyl)guanidine;
10) (2S,3R,4S)-N″-cyano-N-(6-nitro-3,4-dihydro-3-hydroxy-2-methyl-2-dimethoxymethyl-2H-benzopyran-4-yl)-N′-(4-chlorophenyl)guanidine;
11) (2S,3S,4R)-N″-cyano-N-(6-nitro-3,4-dihydro-3-hydroxy-2-methyl-2-dimethoxymethyl-2H-benzopyran-4-yl)-N′-(4-chlorophenyl)guanidine;
12) (2S,3R,4S)-N″-cyano-N-(6-nitro-3,4-dihydro-3-hydroxy-2-methyl-2-dimethoxymethyl-2H-benzopyran-4-yl)-N′-(3-chlorophenyl)guanidine;
13) (2S,3S,4R)-N″-cyano-N-(6-nitro-3,4-dihydro-3-hydroxy-2-methyl-2-dimethoxymethyl-2H-benzopyran-4-yl)-N′-(3-chlorophenyl)guanidine;
14) (2S,3R,4S)-N″-cyano-N-(6-nitro-3,4-dihydro-3-hydroxy-2-methyl-2-dimethoxymethyl-2H-benzopyran-4-yl)-N′-(3-trifluoromethylphenyl)guanidine;
15) (2S,3S,4R)-N″-cyano-N-(6-nitro-3,4-dihydro-3-hydroxy-2-methyl-2-dimethoxymethyl-2H-benzopyran-4-yl)-N′-(3-trifluoromethylphenyl)guanidine;
16) (2S,3R,4S)-N″-cyano-N-(6-nitro-3,4-dihydro-3-hydroxy-2-methyl-2-dimethoxymethyl-2H-benzopyran-4-yl)-N′-(4-methoxyphenyl)guanidine;
17) (2S,3S,4R-N″-cyano-N-(6-nitro-3,4dihydro-N′-(4-methoxyphenyl)guanidine;
18) (2R,3R,4S)-N″-cyano-N-(6-nitro-3,4-dihydro-3-hydroxy-2-methyl-2-dimethoxymethyl-2H-benzopyran-4-yl)-N′-(4-methylphenyl)guanidine;
19) (2R,3S,4R)-N″-cyano-N-(6-nitro-3,4-dihydro-3-hydroxy-2-methyl-2-dimethoxymethyl-2H-benzopyran-4-yl)-N′-(4-methylphenyl)guanidine;
20) (2R,3R,4S)-N″-cyano-N-(6-nitro-3,4-dihydro-3-hydroxy-2-methyl-2-dimethoxymethyl-2H-benzopyran-4-yl)-N′-(4-methoxybenzyl)guanidine;)
21) (2R,3S,4R)-N″-cyano-N-(6-nitro-3,4-dihydro-3-hydroxy-2-methyl-2-dimethoxymethyl-2H-benzopyran-4-yl)-N′-(4-methoxybenzyl)guanidine;
22) (2R,3R,4S)-N″-cyano-N-(6-nitro-3,4-dihydro-3-hydroxy-2-methyl-2-dimethoxymethyl-2H-benzopyran-4-yl)-N′-benzylguanidine;
23) (2R,3S,4R)-N″-cyano-N-(6-nitro-3,4-dihydro-3-hydroxy-2-methyl-2-dimethoxymethyl-2H-benzopyran-4-yl)-N′-benzylguanidine;
24) (2S,3S,4R)-N″-cyano-N-(6-nitro-3,4-dihydro-3-hydroxy-2-methyl-2-dimethoxymethyl-2H-benzopyran-4-yl)-N′-benzylguanidine;
25) (2S,3S,4R)-N″-cyano-N-(6-nitro-3,4-dihydro-3-hydroxy-2-methyl-2-dimethoxymethyl-2H-benzopyran-4yl)-benzylguanidine;
26) (2R,3R,4S)-N″-cyano-N-(3,4-dihydro-3-hydroxy-2-methyl-2-dimethoxymethyl-2H-benzopyran-4-yl)-N′-(4-chlorophenyl)guanidine;
27) (2R,3S,4R)-N″-cyano-N-(3,4-dihydro-3-hydroxy-2-methyl-2-dimethoxymethyl-2H-benzopyran-4-yl)-N′-(4-chlorophenyl)guanidine;
28) (2R,3R,4S)-N″-cyano-N-(6-nitro-3,4-dihydro-3-hydroxy-2-methyl-2-hydroxymethyl-2H-benzopyran-4-yl)-N′-(4-chlorophenyl)guanidine;
29) (2R,3S,4R)-N″-cyano-N-(6-nitro-3,4-dihydro-3-hydroxy-2-methyl-2-hydroxymethyl-2H-benzopyran-4-yl)-N′-(4-chlorophenyl)guanidine;
30) (2R,3R,4S)-N″-cyano-N-(6-nitro-3,4-dihydro-3-hydroxy-2-methyl-2-methoxymethyl-2H-benzopyran-4-yl)-N′-(4-chlorophenyl)guanidine;
31) (2R,3S,4R)-N″-cyano-N-(6-nitro-3,4-dihydro-3-hydroxy-2-methyl-2-methoxymethyl-2H-benzopyran-4-yl)-N′-(4-chlorophenyl)guanidine;
32) (2S,3R,4S)-N″-cyano-N-(6-nitro-3,4-dihydro-3-hydroxy-2-methyl-2-dimethoxymethyl-2H-benzopyran-4-yl)-N′-(2-chlorophenyl)guanidine;
33) (2S,3S,4R)-N″-cyano-N-(6-nitro-3,4dihydro-3-hydroxy-2-dimethoxymethyl-2H-benzopyran-4-yl)-N′-(2-chlorophenyl)guanidine;
34) (2S,3R,4S)-N″-cyano-N-(6-nitro-3,4-dihydro-3-hydroxy-2-methyl-2-dimethoxymethyl-2H-benzopyran-4-yl)-N′-(2-trifluoromethylphenyl)guanidine;
35) (2S,3S,4R)-N″-cyano-N-(6-nitro-3,4-dihydro-3-hydroxy-2-methyl-2-dimethoxymethyl-2H-benzopyran-4-yl)-N′-(2-trifluoromethylphenyl)guanidine;
36) (2S,3S,4S)-N″-cyano-N-(6-nitro-3,4-dihydro-3-hydroxy-2-methyl-2-dimethoxymethyl-2H-benzopyran-4-yl)-N′-(2-chlorobenzyl)guanidine;
37) (2S,3S,4R)-N″-cyano-N-(6-nitro-3,4-dihydro-3-hydroxy-2-methyl-2-dimethoxymethyl-2H-benzopyran-4-yl)-N′-(2-chlorobenzyl)guanidine;
38) (2S,3S,4R)-N″-cyano-N-(6-nitro-3,4-dihydro-3-acetoxy-2-methyl-2-dimethoxymethyl-2H-benzopyran-4-yl)-N′-benzylguanidine;
39) (2S)-N″-cyano-N-(6-nitro-2-methyl-2-dimethoxymethyl -2-H-benzopyran-4-yl)-N′-benzylguanidine;
40) (2S,3S,4R)-N″-cyano-N-(6-amino-3,4-dihydro-3-hydroxy-2-methyl-2-dimethoxymethyl-2H-benzopyran-4-yl)-N′-benzylguanidine;
41) (2S,3S,4R)-N″-cyano)-N-(6-acetoxyamino-3,4-dihydro-3-hydroxy-2-methyl-2-dimethoxymethyl-2H-benzopyran-4-yl)-N′-benzylguanidine;
42) (2S,3S,4R)-N″-cyano-N-(6-methanesulfonylamino-3,4-dihydro-3-hydroxy-2-methyl-2-dimethoxymethyl-2H-benzopyran-4-yl)-N′-benzylguanidine;
43) (2S,3S,4R)-N″-cyano-N-(6-cyano-3,4-dihydro-3-hydroxy-2-methyl-2-dimethoxymethyl-2H-benzopyran-4-yl)-N′-(4-chlorophenyl)guanidine;
44) (2S,3R,4S)-N″-cyano-N-(6-cyano-3,4-dihydro-3-hydroxy-2-methyl-2-dimethoxymethyl-2H-benzopyran-4-yl)-N′-(4-chlorophenyl)guanidine;
45) (2S,3S,4R)-N″-cyano-N-(6-cyano-3,4-dihydro-3-hydroxy-2-methyl-2-dimethoxymethyl-2H-benzopyran-4-yl)-N′-benzylguanidine;
46) (2S,3R,4S)-N″-cyano-N-(6-cyano-3,4-dihydro-3-hydroxy-2-methyl-2-dimethoxymethyl-2H-benzopyran-4-yl)-N′-benzylguanidine;
47) (2S,3S,4R)-N″-cyano-N-(6-bromo-3,4-dihydro-3-hydroxy-2-methyl-2-dimethoxymethyl-2H-benzopyran-4-yl)-N′-benzylguanidine;
48) (2S,3S,4R)-N″-cyano-N-(6-nitro-3,4-dihydro-3-hydroxy-2-methyl-2-(dimethoxymethyl-2H-benzopyran-4-yl)-N′-(3,4-dimethoxybenzyl)guanidine;
49) (2S,3S,4R)-N″-cyano-N-(6-amino-3,4-dihydro-3-hydroxy-2-methyl-2-(dimethoxymethyl-2H-benzopyran-4-yl)-N′-(3,4-dimethoxylbenzyl)guanidine;
50) (2S,3S,4R)-N″-cyano-N-(6-nitro-3,4-dihydro-3-hydroxy-2-methyl-2-dimethoxymethyl-2H-benzopyran-4-yl)-N′-(4-methoxybenzyl)guanidine;
51) (2S,3S,4R)-N″-cyano-N-(6-amino-3,4-dihydro-3-hydroxy-2-methyl-2-dimethoxymethyl-2H-benzopyran-4-yl)-N′-(4-methoxybenzyl)guanidine;
52) (2S,3S,4R)-N″-cyano-N-(6-nitro-3,4-dihydro-3-hydroxy-2-methyl-2-dimethoxymethyl-2H-benzopyran-4-yl)-N′-(3-nitrobenzyl)guanidine;
53) (2S,3S,4R)-N″-cyano-N-(6-nitro-3,4-dihydro-3-hydroxy-2-methyl-2-dimethoxymethyl-2H-benzopyran-4-yl)-N′-(3-trifluoromethylbenzyl)guanidine;
54) 2S,3S,4R)-N″-cyano-N-(6-amino-3,4-dihydro-3-hydroxy-2-methyl-2-dimethoxymethyl-2H-benzopyran-4-yl)-N′-(3-trifluoromethylbenzyl)guanidine;
55) (2S,3S,4R)-N″-cyano-N-(6-methanesulfonyloxy-3,4-dihydro-3-hydroxy-2-methyl-2-dimethoxymethyl-2H-benzopyran-4-yl)-N′-benzylguanidine;
56) (2R,3S,4R)-N″-cyano-N-(6-methanesulfonyloxy-3,4-dihydro-3-hydroxy-2-methyl-2-dimethoxymethyl-2H-benzopyran-4-yl)-N′-benzylguanidine;
57) (2S,3R,4S)-N″-cyano-N-(6-amino-3,4-dihydro-3-hydroxy-2-methyl-2-dimethoxymethyl-2!!-benzopyran-4-yl)-N′-benzylguanidine;
58) (2R,3R,4S)-N″-cyano-N-(6-amino-3,4-dihydro-3-hydroxy-2-methyl-2-dimethoxymethyl-2H-benzopyran-4-yl)-N′-benzylguanidine;
59) (2R,3S,4R)-N″-cyano-N-(6-amino-3,4-dihydro-3-hydroxy-2-methyl-2-dimethoxymethyl-2H-benzopyran-4-yl)-N′-benzylguanidine;
60) (2S,3S,4R)-N″-cyano-N-(6-nitro-3,4-dihydro-3-hydroxy-2-methyl-2-([1,3]dioxolan-2-yl)-2H-benzopyran-4-yl)-N′-benzylguanidine;
61) (2S,3S,4R)-N″-cyano-N-(6-amino-3,4-dihydro-3-hydroxy-2-methyl-2-([1,3]dioxolan-2-yl)-2H-benzopyran-4-yl)-N′-benzylguanidine;
62) (2S,3S,4R)-N″-cyano-N-(6-nitro-3,4-dihydro-3-hydroxy-2-methyl-2-([1,3]dioxan-2-yl)-2H-benzopyran-4-yl)-N′-benzylguanidine;
63) (2S,3S,4R)-N″-cyano-N-(6-amino-3,4-dihydro-3-hydroxy-2-methyl-2-([1,3]dioxan-2-yl)-2H-benzopyran-4-yl)-N′-benzylguanidine;
64) (2S,3S,4R)-N″-cyano-N-(6-nitro-3,4-dihydro-3-hydroxy-2-methyl-2-([1,3]-5,5-dimethyldioxan-2-yl)-2H-benzopyran-4-yl)-N′-benzylguanidine;
65) (2S,3S,4R)-N″-cyano-N-(6-amino)-3,4-dihydro-3-hydroxy-2-methyl-2-([1,3]-5,5-dimethyldioxan-2-yl)-2H-benzopyran-4-y)-N′-benzylguanidine;
66) (2S,3S,4R)-N″-cyano-N-(6-nitro-3,4-dihydro-3-hydroxy-2-methyl-2-diethoxymethyl-2H-benzopyran-4-yl)-N′-benzylguanidine;
67) (2S,3S,4R)-N″-cyano-N-(6-amino-3,4-dihydro-3-hydroxy-2-methyl-2-diethoxymethyl-2H-benzopyran-4-yl)-N′-benzylguanidine;
68) (2S,3S,4R)-N″-cyano-N-(6-methoxycarbonyl-3,4-dihydro-3-hydroxy-2-methyl-2-dimethoxymethyl-2H-benzopyran-4-yl)-N′-benzylguanidine;
69) (2R,3S,4R)-N″-cyano-N-(6-methoxycarbonyl-3,4-dihydro-3-hydroxy-2-methyl-2-dimethoxymethyl-2H-benzopyran-4-yl)-N′-benzylguanidine;
70) (3S,4R)-N″-cyano-N-(8-nitro-3,4-dihydro-3-hydroxy-2-methyl-2-dimethoxymethyl-2H-benzopyran-4-yl)-N′-benzylguanidine;
71) (2S,3S,4R)-N″-cyano-N-(8-amino-3,4-dihydro-3-hydroxy-2-methyl-2-dimethoxymethyl-2H-benzopyran-4-yl)-N′-benzylguanidine;
72) (2R,3S,4R)-N″-cyano-N-(8-amino-3,4-dihydro-3-hydroxy-2-methyl-2-dimethoxymethyl-2H-benzopyran-4-yl)-N′-benzylguanidine; and
73) (2S,3S,4R)-N″-cyano-N-(6-amino-3,4-dihydro-3-hydroxy-2-methyl-2-dimethoxymethyl-2H-benzopyran-4-yl)-N′-benzylguanidine.

5. The formulation of claim 1, wherein the compound of formula 1 is (2S,3S,4R)-N″-cyano-N-(6-amino-3,4-dihydro-3-hydroxy-2-methyl-2-dimethoxymethyl-2H-benzopyran-4-yl)-N′-benzylguanidine.

6. The formulation of claim 1, wherein the microparticles have an average diameter of about 0.5 to about 20 microns.

7. The formulation of claim 1, wherein the nanoparticles have an average diameter of about 0.5 microns or less

8. The formulation of claim 1, comprising:
about 1 to about 30% by weight of a compound of formula 1;
about 0.01 to about 30% by weight of a buffer;
about 0.01 to about 10% by weight of a chelator;
about 0.001 to about 10% by weight of a preservative;
about 0.1 to about 20% by weight of an isotonizer; and
about 0 to about 20% by weight of a solubilizing agent, suspending agent, surfactant, or thickening agent.

9. The formulation of claim 1, comprising:
about 1 to about 30% by weight of a compound of formula 1;
about 0.01 to about 10% by weight of monobasic sodium phosphate monohydrate;
about 0.05 to about 20% by weight disodium phosphate heptahydrate;
about 0.01 to about 10% by weight of edetate disodium;
about 0.001 to about 10% by weight of benzalkonium chloride;
about 0.1 to about 20% by weight of sodium chloride;
about 0 to about 20% by weight of Polysorbate 80; and
water.

10. The formulation of claim 9, comprising:
about 1 to about 15% by weight of a compound of formula 1;
about 0.01 to about 1% by weight of monobasic sodium phosphate monohydrate;
about 0.05 to about 2% by weight disodium phosphate heptahydrate;
about 0.01 to about 1% by weight of edetate disodium;
about 0.005 to about 0.1% by weight of benzalkonium chloride;
about 0.1 to about 2% by weight of sodium chloride;
about 0.01 to about 5% by weight of Polysorbate 80; and
water.

11. The formulation of claim 9, comprising:
about 1 to about 10% by weight of a compound of formula 1;
about 0.01 to about 0.05% by weight of monobasic sodium phosphate monohydrate;
about 0.1 to about 0.5% by weight disodium phosphate heptahydrate;
about 0.01 to about 0.1% by weight of edetate disodium;
about 0.005 to about 0.05% by weight of benzalkonium chloride;
about 0.5 to about 1% by weight of sodium chloride;
about 0.01 to about 0.1% by weight of Polysorbate 80; and
water.

12. The formulation of claim 1, comprising:
about 1 to about 30% by weight of a compound of formula 1;
about 0.01 to about 10% by weight of monobasic sodium phosphate monohydrate;
about 0.05 to about 20% by weight disodium phosphate heptahydrate;
about 0.01 to about 10% by weight of edetate disodium;
about 0.001 to about 10% by weight of benzalkonium chloride;
about 0.1 to about 20% by weight of sodium chloride;
about 0 to about 20% by weight of Povidone K29-32; and
water.

13. The formulation of claim 12, comprising:
about 1 to about 15% by weight of a compound of formula 1;
about 0.01 to about 1% by weight of monobasic sodium phosphate monohydrate;
about 0.05 to about 2% by weight disodium phosphate heptahydrate;
about 0.01 to about 1% by weight of edetate disodium;
about 0.001 to about 0.5% by weight of benzalkonium chloride;
about 0.1 to about 5% by weight of sodium chloride;
about 0 to about 10% by weight of Povidone K29-32; and
water.

14. The formulation of claim 12, comprising:
about 1 to about 10% by weight of a compound of formula 1;
about 0.01 to about 0.05% by weight of monobasic sodium phosphate monohydrate;
about 0.1 to about 0.5% by weight disodium phosphate heptahydrate;
about 0.01 to about 0.1% by weight of edetate disodium;
about 0.005 to about 0.05% by weight of benzalkonium chloride;
about 0.5 to about 1% by weight of sodium chloride;
about 1 to about 5% by weight of Povidone K29-32; and
water.

15. A topical pharmaceutical formulation comprising:
about 1 to about 10% by weight of (2S,3S,4R)-N″-cyano-N-(6-amino-3,4-dihydro-3-hydroxy-2-methyl-2-dimethoxymethyl-2H-benzopyran-4-yl)-N′-benzylguanidine;
about 0.01 to about 0.05% by weight monobasic sodium phosphate monohydrate;
about 0.1 to about 0.5% by weight disodium phosphate heptahydrate;
about 0.01 to 0.1% by weight edetate disodium;
about 0.01 to about 0.05% by weight benzalkonium chloride;
about 0.1 to about 1% by weight sodium, chloride;
about 0.01 to 0.1% by weight Polysorbate 80; and
water,
wherein the (2S,3S,4R)-N″-cyano-N-(6-amino-3,4-dihydro-3-hydroxy-2-methyl-2-dimethoxymethyl-2H-benzopyran-4-yl)-N′-benzylguanidine is provided as microparticles having an average diameter of about 1 to about 10 microns.

16. The formulation of claim 15, comprising
about 3% to about 10% by weight of (2S,3S,4R)-N″-cyano-N-(6-amino-3,4-dihydro-3-hydroxy-2-methyl-2-dimethoxymethyl-2H-benzopyran-4-yl)-N′-benzylguanidine;
about 0.02 to about 0.04% by weight monobasic sodium phosphate monohydrate;
about 0.1 to about 0.3% by weight disodium phosphate heptahydrate;
about 0.04 to 0.06% by weight edetate disodium;
about 0.01 to about 0.03% by weight benzalkonium chloride;
about 0.5 to about 1% by weight sodium, chloride;
about 0.03 to 0.5% by weight Polysorbate 80; and
water.

17. The formulation of claim 15, comprising:
about 3% by weight of (2S,3S,4R)-N″-cyano-N-(6-amino-3,4-dihydro-3-hydroxy-2-methyl-2-dimethoxymethyl-2H-benzopyran-4-yl)-N′-benzylguanidine;
about 0.031% by weight monobasic sodium phosphate monohydrate;
about 0.203% by weight disodium phosphate heptahydrate;
about 0.049% by weight edetate disodium;
about 0.02% by weight benzalkonium chloride;
about 0.881% by weight sodium, chloride;
about 0.44% by weight Polysorbate 80; and
water,
wherein the (2S,3S,4R)-N″-cyano-N-(6-amino-3,4-dihydro-3-hydroxy-2-methyl-2-dimethoxymethyl-2H-benzopyran-4-yl)-N′-benzylguanidine is provided as microparticles having an average diameter of about 1 to about 10 microns.

18. The formulation of claim 15, comprising:
about 10% by weight (2S,3S,4R)-N″-cyano-N-(6-amino-3,4-dihydro-3-hydroxy-2-methyl-2-dimethoxymethyl-2H-benzopyran-4-yl)-N′-benzylguanidine;
about 0.029% by weight monobasic sodium phosphate monohydrate;
about 0.193% by weight disodium phosphate heptahydrate;
about 0.047% by weight edetate disodium;
about 0.019% by weight benzalkonium chloride;
about 0.837% by weight sodium, chloride;
about 0.042% by weight Polysorbate 80; and
and water.

19. A topical pharmaceutical formulation comprising:
about 1 to about 10% by weight of (2S,3S,4R)-N″-cyano-N-(6-amino-3,4-dihydro-3-hydroxy-2-methyl-2-dimethoxymethyl-2H-benzopyran-4-yl)-N′-benzylguanidine;
about 0.01 to about 0.05% by weight monobasic sodium phosphate monohydrate;
about 0.1 to about 0.5% by weight disodium phosphate heptahydrate;
about 0.01 to 0.1% by weight edetate disodium;
about 0.01 to about 0.05% by weight benzalkonium chloride;
about 0.1 to about 1% by weight sodium, chloride;
0 to about 5% by weight Polysorbate 80;
0 to about 5% by weight Povidone K29-32; and
water,
wherein the (2S,3S,4R)-N″-cyano-N-(6-amino-3,4-dihydro-3-hydroxy-2-methyl-2-dimethoxymethyl-2H-benzopyran-4-yl)-N′-benzylguanidine is provided as nanoparticles having an average diameter of less than 0.3 microns.

20. The formulation of claim 19, comprising:
about 0.01 to about 0.05% by weight monobasic sodium phosphate monohydrate;
about 0.1 to about 0.5% by weight disodium phosphate heptahydrate;
about 0.01 to 0.1% by weight edetate disodium;
about 0.01 to about 0.05% by weight benzalkonium chloride;
about 0.1 to about 1% by weight sodium, chloride; and
about 1 to about 5% by weight Polysorbate 80.

21. The formulation of claim 19, comprising:
about 0.01 to about 0.05% by weight monobasic sodium phosphate monohydrate;
about 0.1 to about 0.5% by weight disodium phosphate heptahydrate;
about 0.01 to 0.1% by weight edetate disodium;
about 0.01 to about 0.05% by weight benzalkonium chloride;
about 0.1 to about 1% by weight sodium, chloride; and
about 1 to about 5% by weight Povidone K29-32.

22. A method of reducing intraocular pressure in the eye of a patient on need thereof, comprising topically applying the formulation of claim 1 to the eye of a patient in need thereof.

23. A method of treating glaucoma, comprising administering to a patient in need thereof
